(19) United States
(12) Patent Application Publication
(10) Pub. No.: US 2025/0051251 A1
(43) Pub. Date: Feb. 13, 2025

(54) PROCESS FOR THE PREPARATION AND PURIFICATION OF TRIFLUOROETHYLENE

(71) Applicant: Arkema France, Colombes (FR)

(72) Inventors: Jérémy RETALI, PIERRE-BENITE CEDEX (FR); Kevin HISLER, PIERRE-BENITE CEDEX (FR); Cédric LAVY, PIERRE-BENITE CEDEX (FR)

(21) Appl. No.: 18/718,469

(22) PCT Filed: Dec. 13, 2022
(86) PCT No.: PCT/FR2022/052338

§ 371 Date: Jun. 11, 2024

(30) Foreign Application Priority Data
Dec. 23, 2021 (FR) FR2114370

(51) Int. Cl. C07C 17/38; B01D 53/22; B01D 71/16; B01D 71/26; B01D 71/34; B01D 71/52; B01D 71/64; B01D 71/70

(52) U.S. Cl. CPC C07C 17/38; B01D 53/226; B01D 53/228; B01D 71/16; B01D 71/26; B01D 71/262; B01D 71/34; B01D 71/5223; B01D 71/64; B01D 71/70

(57) ABSTRACT

The present invention relates to a process for the purification of a fluorocarbon from a mixture comprising said fluorocarbon and hydrogen, said process comprising a stage of bringing said mixture into contact with a membrane M1 to form a stream F1 comprising the fluorocarbon and a stream F2 comprising the hydrogen. The present invention also relates to a process for the production of trifluoroethylene. The present invention also relates to a process for the separation of a hydrofluoroolefin or of a hydrofluoroalkane from nitrogen by membrane separation. The present invention also relates to a process for the separation of trifluoroethylene from chlorotrifluoroethylene or from a hydrofluorocarbon.

### TECHNICAL FIELD OF THE INVENTION

The present invention relates to a process for the production and purification of hydrofluoroolefins. In particular, the present invention relates to a process for the production of trifluoroethylene (VF₃) and to the purification of the latter.

### TECHNOLOGICAL BACKGROUND OF THE INVENTION

Fluoro-olefins, such as VF₃, are known and are used as monomers or comonomers for the manufacture of fluorocarbon polymers exhibiting noteworthy characteristics, in particular excellent chemical resistance and good thermal resistance.

Trifluoroethylene is a gas under standard conditions of pressure and temperature. The main risks associated with the use of this product relate to its flammability, its propensity for self-polymerization when it is not stabilized, its explosiveness due to its chemical instability and its supposed sensitivity to peroxidation, by analogy with other halogenated olefins. Trifluoroethylene exhibits the distinguishing feature of being extremely flammable, with a lower explosive limit (LEL) of approximately 10% and an upper explosive limit (UEL) of approximately 30%. The major hazard, however, is associated with the propensity of VF₃ to decompose violently and explosively under certain pressure conditions in the presence of an energy source, even in the absence of oxygen.

Given the main risks above, the synthesis and also the storage of VF₃ pose particular problems and impose strict safety rules throughout these processes. A known route for the preparation of trifluoroethylene uses, as starting materials, chlorotrifluoroethylene (CTFE) and hydrogen in the presence of a catalyst and in the gas phase. A process for the production of trifluoroethylene by hydrogenolysis of CTFE in the gas phase and in the presence of a catalyst based on a metal from group VIII at atmospheric pressure and at relatively low temperatures is known from WO2013/128102. In this document, the purification of the crude mixture resulting from the reaction and comprising trifluoroethylene consists of a series of washing and distillation stages. In particular, gases, such as hydrogen and nitrogen, are separated from the VF₃ using an absorption column fed with ethanol. Hydrogen and nitrogen exit at the column top, while the products of the reaction (VF₃, CTFE, and the like) are dissolved in the ethanol and are directed to a desorption section in order to separate them from the ethanol. This stage requires the use of a large amount of ethanol, which impacts the environmental balance of the overall process (requires treatment of the solvent used, high energy cost). In addition, compounds such as trifluoroethylene and chlorotrifluoroethylene can be difficult to separate. There is thus a need for the employment of a more efficient and more environmentally friendly process.

### SUMMARY OF THE INVENTION

According to a first aspect, the present invention relates to a process for the purification of a fluorocarbon from a mixture comprising said fluorocarbon and hydrogen, said process comprising a stage (a) of bringing said mixture into contact with a membrane M1 to form a stream F1 comprising the fluorocarbon and a stream F2 comprising the hydrogen.

The present invention makes possible the employment of a more efficient and more environmentally friendly process. This is because the use of a membrane as described in the present patent application exhibits advantages of efficiency and of compatibility with the environment in view of the elimination of the absorption solvent ordinarily used to separate a fluorocarbon from hydrogen. The present invention also exhibits advantages in terms of production cost (absence of treatment of the waste solvent) and of simplification of the process. According to a preferred embodiment, said membrane M1 is made of a material selected from the group consisting of polyolefin, polyether, polyimide, polyaramid, polyamide, polysulfone, polyvinylidene fluoride, cellulose, polymethyl methacrylate, polytetrafluoroethylene, polyvinyl fluoride, polychlorotrifluoroethylene, polyethylene-tetrafluoroethylene and tetrafluoroethylene/perfluorovinyl ether copolymer optionally substituted by an SO₃H group. According to a preferred embodiment, the fluorocarbon is selected from the group consisting of fluoromethane, difluoromethane, trifluoromethane, trifluoroethylene, 1,1-difluoroethylene, 1,2-difluoroethylene, tetrafluoroethylene, chlorotrifluoroethylene, fluoroethane, pentafluoroethane, 1,1,1,2-tetrafluoroethane, 1,1,2,2-tetrafluoroethane, 1,1-difluoroethane, 1,2-difluoroethane, 1,1,2-trifluoroethane, 1,1,1-trifluoroethane, 2-chloro-1,1,2-trifluoroethane, 1-chloro-1,1,2-trifluoroethane, 2-chloro-1,1,1-trifluoroethane, 3,3,3-trifluoropropene, hexafluoropropene, 1,1,1,3,3,3-hexafluoropropane, 1,1,2,2,3,3-hexafluoropropane, 1,1,1,2,2,3-hexafluoropropane, 1,1,1,2,3,3-hexafluoropropane, 1,1,2,2,3-pentafluoropropane, 1,1,1,2,2-pentafluoropropane, 1,1,2,3,3-pentafluoropropane, 1,1,1,2,3-pentafluoropropane, 1,1,1,3,3-pentafluoropropane, 2-chloro-1,1,1,2-tetrafluoropropane, 3-chloro-1,1,1,3-tetrafluoropropane, 2,3-dichloro-1,1,1-trifluoropropane, 1,1,3,3,3-pentafluoropropene, 1,1,2,3,3-pentafluoropropene, 1,2,3,3,3-pentafluoropropene, 2-chloro-3,3,3-trifluoropropene, 1-chloro-3,3,3-trifluoropropene, 2,3,3,3-tetrafluoropropene, 1,3,3,3-tetrafluoropropene, 1,1,2,3-tetrafluoropropene, 1,1,3,3-tetrafluoropropene, 1,2,3,3-tetrafluoropropene, 1,1,3-trifluoropropene, 1,1,2-trifluoropropene, 3,3,3-trifluoropropene, 1,2,3-trifluoropropene, 2,3,3-trifluoropropene, 1,3,3-trifluoropropene, 1,1-difluoropropene, 1,2-difluoropropene, 2,3-difluoropropene and 3,3-difluoropropene.

According to a preferred embodiment, said fluorocarbon is selected from the group consisting of trifluoroethylene, 1,1-difluoroethylene, 1,2-difluoroethylene, tetrafluoroethylene, chlorotrifluoroethylene, pentafluoroethane, 1,1,1,2-tetrafluoroethane, 1,1,2,2-tetrafluoroethane, 1,1-difluoroethane, 1,2-difluoroethane, 1,1,2-trifluoroethane, 2-chloro-1,1,2-trifluoroethane, 1-chloro-1,1,2-trifluoroethane, 2-chloro-1,1,1-trifluoroethane, difluoromethane, trifluoromethane, 2,3,3,3-tetrafluoropropene, 1,3,3,3-tetrafluoropropene, 1-chloro-3,3,3-trifluoropropene, 2-chloro-3,3,3-trifluoropropene, 1,1,1,2,2-pentafluoropropane, 1,1,1,2,3-pentafluoropropane, 1,1,1,3,3-pentafluoropropane, 3-chloro-1,1,1,3-tetrafluoropropane, 2-chloro-1,1,1,2-tetrafluoropropane, 2,3-dichloro-1,1,1-trifluoropropane, hexafluoropropene, 1,2,3,3,3-pentafluoropropene, 3,3,3-trifluoropropene and 1,1,1,2,3,3-hexafluoropropane.

According to a preferred embodiment, said fluorocarbon is selected from the group consisting of trifluoroethylene, 1,1-difluoroethylene, 1,2-difluoroethylene, tetrafluoroethylene, pentafluoroethane, 1,1,1,2-tetrafluoroethane, 1,1,2,2-tetrafluoroethane, 1,1-difluoroethane, 1,2-difluoroethane, 1,1,2-trifluoroethane, difluoromethane, trifluoromethane, 2,3,3,3-tetrafluoropropene, 1,3,3,3-tetrafluoropropene, 1,1,1,2,2-pentafluoropropane, 1,1,1,2,3-pentafluoropropane, 1,1,1,3,3-pentafluoropropane, hexafluoropropene, 1,2,3,3,3-pentafluoropropene, 3,3,3-trifluoropropene and 1,1,1,2,3,3-hexafluoropropane.

According to a preferred embodiment, said membrane M1 has a selectivity of greater than 5, said selectivity being calculated by the ratio of the permeability of the hydrogen to the permeability of said fluorocarbon through said membrane M1.

According to a preferred embodiment, said membrane M1 is made of a material selected from the group consisting of polypropylene, polymethylpentene, poly[oxy(2,6-dimethyl-1,4-phenylene)], poly(phenylene oxide), polyvinylidene fluoride, cellulose and polyimide.

According to a preferred embodiment, said mixture and said stream F1 also comprise nitrogen, said process comprising a stage (b) of bringing said stream F1 into contact with a membrane M1′ to form a stream F3 comprising said fluorocarbon and a stream F4 comprising nitrogen.

According to a preferred embodiment, said membrane M1′ is made of a material selected from the group consisting of polypropylene, polymethylpentene or polyalkylsiloxane.

According to a second aspect, the present invention provides a process for the separation of a mixture comprising a hydrofluoroolefin and nitrogen, said process comprising a stage of bringing said mixture into contact with a membrane M3 to form a stream F7 comprising said hydrofluoroolefin and a stream F8 comprising the nitrogen, said membrane M3 being made of a material containing a siloxane functional group.

According to a preferred embodiment, said membrane M3 is made of a material containing a functional group of formula —[—(R)(R′)Si—O]ₙ— with R and R′ independently selected from the group consisting of hydrogen, C₁-C₂₀ alkyl, C₃-C₁₀ cycloalkyl, C₆-C₁₂ aryl and n being an integer greater than 50, preferably greater than 100, in particular greater than 1000.

According to a preferred embodiment, said membrane M3 is made of polyalkylsiloxane. According to a preferred embodiment, said membrane M3 is made of polydimethylsiloxane. According to a preferred embodiment, said hydrofluoroolefin is selected from the group consisting of trifluoroethylene, 1,1-difluoroethylene, 1,2-difluoroethylene, tetrafluoroethylene, 3,3,3-trifluoropropene, hexafluoropropene, 1,1,3,3,3-pentafluoropropene, 1,1,2,3,3-pentafluoropropene, 1,2,3,3,3-pentafluoropropene, 2,3,3,3-tetrafluoropropene, 1,3,3,3-tetrafluoropropene, 1,1,2,3-tetrafluoropropene, 1,1,3,3-tetrafluoropropene, 1,2,3,3-tetrafluoropropene, 1,1,3-trifluoropropene, 1,1,2-trifluoropropene, 3,3,3-trifluoropropene, 1,2,3-trifluoropropene, 2,3,3-trifluoropropene, 1,3,3-trifluoropropene, 1,1-difluoropropene, 1,2-difluoropropene, 2,3-difluoropropene and 3,3-difluoropropene.

According to a preferred embodiment, said hydrofluoroolefin is selected from the group consisting of trifluoroethylene, 1,1-difluoroethylene, 1,2-difluoroethylene, tetrafluoroethylene, 2,3,3,3-tetrafluoropropene, 1,3,3,3-tetrafluoropropene, hexafluoropropene, 1,2,3,3,3-pentafluoropropene and 3,3,3-trifluoropropene.

According to a third aspect, the present invention provides a process for the separation of a mixture comprising a hydrofluoroalkane and nitrogen, said process comprising a stage of bringing said mixture into contact with a membrane M3′ to form a stream FT comprising said hydrofluoroalkane and a stream F8′ comprising the nitrogen, said membrane M3′ being made of polyolefin.

According to a preferred embodiment, said membrane M3′ is made of a material selected from the group consisting of polyethylene, polypropylene, polymethylpentene, polyhexene, polypentene and polybutene.

According to a preferred embodiment, said hydrofluoroalkane is selected from the group consisting of pentafluoroethane, 1,1,1,2-tetrafluoroethane, 1,1,2,2-tetrafluoroethane, 1,1-difluoroethane, 1,2-difluoroethane, 1,1,2-trifluoroethane, fluoromethane, difluoromethane, trifluoromethane, 1,1,1,2,2-pentafluoropropane, 1,1,1,2,3-pentafluoropropane, 1,1,1,3,3-pentafluoropropane, 1,1,1,2,3,3-hexafluoropropane and 3,3,3-trifluoropropene.

According to a fourth aspect, the present invention provides a process for the production of trifluoroethylene in a reactor furnished with a fixed catalytic bed comprising a catalyst, said process comprising a stage A) of reaction of chlorotrifluoroethylene with hydrogen in the presence of the catalyst and in the gas phase to produce a stream comprising the trifluoroethylene, chlorotrifluoroethylene and unreacted hydrogen and a stage B) of bringing a stream comprising trifluoroethylene, chlorotrifluoroethylene and possibly hydrogen into contact with a membrane M2 to form a stream F5 comprising trifluoroethylene and possibly hydrogen and a stream F6 comprising chlorotrifluoroethylene and possibly hydrogen.

According to a preferred embodiment, said membrane M2 is made of a material selected from the group consisting of polyolefin, polyether, polyimide, polymethyl methacrylate, cellulose and polyvinylidene fluoride.

According to a preferred embodiment, said membrane M2 is made of a material selected from the group consisting of polypropylene, polymethylpentene, poly[oxy(2,6-dimethyl-1,4-phenylene)], poly(phenylene oxide), polyimide and cellulose acetate.

According to a preferred embodiment, said membrane M2 has a selectivity of greater than 9, said selectivity being calculated by the ratio of the permeability of the hydrogen to the permeability of the trifluoroethylene through said membrane M2, and said membrane M2 has a selectivity of greater than 20, said selectivity being calculated by the ratio of the permeability of chlorotrifluoroethylene to the permeability of the trifluoroethylene through said membrane M2.

According to a particular embodiment, said membrane M2 is made of polypropylene or polymethylpentene.

According to another preferred embodiment, said membrane M2 has a selectivity of greater than 100, said selectivity being calculated by the ratio of the permeability of the hydrogen to the permeability of the chlorotrifluoroethylene through said membrane M2, and said membrane M2 has a selectivity of greater than 10, said selectivity being calculated by the ratio of the permeability of the trifluoroethylene to the permeability of the chlorotrifluoroethylene through said membrane M2.

According to a particular embodiment, said membrane M2 is made of polyimide or cellulose acetate.

According to a preferred embodiment, said catalyst comprises from 0.01% to 5% by weight of palladium supported on alumina; preferably, the alumina comprises at least 90% of α-alumina.

According to a preferred embodiment, said stage A) is carried out at a temperature of the fixed catalytic bed of between 50° C. and 250° C.

According to a preferred embodiment, stage B) is carried out at a temperature of 0° C. to 150° C., advantageously of 0° C. to 125° C., preferably of 5° C. to 100° C.

According to this fourth aspect, the present invention also provides a process for the separation of a mixture comprising trifluoroethylene and chlorotrifluoroethylene, said process comprising a stage of bringing said mixture into contact with a membrane M2 to form a stream F5 comprising the trifluoroethylene and a stream F6 comprising the chlorotrifluoroethylene, said membrane M2 being made of a material selected from the group consisting of polyolefin, polyether, polyimide, polymethyl methacrylate, cellulose and polyvinylidene fluoride.

According to a preferred embodiment, said membrane M2 is made of a material selected from the group consisting of polypropylene, polymethylpentene, poly[oxy(2,6-dimethyl-1,4-phenylene)], poly(phenylene oxide), polyimide and cellulose acetate.

According to a fifth aspect, the present invention provides a process for the separation of a mixture comprising trifluoroethylene and a hydrofluorocarbon, said process comprising a stage of bringing said mixture into contact with a membrane M4 to form a stream F9 comprising trifluoroethylene and a stream F10 comprising said hydrofluorocarbon.

According to a preferred embodiment, said membrane M4 is made of a material selected from the group consisting of polyolefin, polyether, polyimide, polyaramid, polyamide, polysulfone, polyvinylidene fluoride, cellulose, polymethyl methacrylate, polytetrafluoroethylene, polyvinyl fluoride, polychlorotrifluoroethylene, polyethylene-tetrafluoroethylene and tetrafluoroethylene/perfluorovinyl ether copolymer optionally substituted by an SO₃H group.

According to a preferred embodiment, said membrane M4 is chosen from a film, a laminated structure, hollow fibers and coated fibers.

According to a preferred embodiment, said membrane M4 is made of a material selected from the group consisting of polyolefin, polyether, polyimide and cellulose.

According to a preferred embodiment, said membrane M4 is made of a material selected from the group consisting of polypropylene, polymethylpentene, cellulose acetate, polyimide, poly[oxy(2,6-dimethyl-1,4-phenylene)] and poly(phenylene oxide).

According to a preferred embodiment, said membrane M4 is made of a material selected from the group consisting of polypropylene and of polymethylpentene.

According to a preferred embodiment, said hydrofluorocarbon is a hydrofluoroalkane selected from the group consisting of pentafluoroethane, 1,1,1,2-tetrafluoroethane, 1,1,2,2-tetrafluoroethane, 1,1-difluoroethane, 1,2-difluoroethane, 1,1,2-trifluoroethane, fluoromethane, difluoromethane, trifluoromethane, 1,1,1,2,2-pentafluoropropane, 1,1,1,2,3-pentafluoropropane, 1,1,1,3,3-pentafluoropropane and 1,1,1,2,3,3-hexafluoropropane. Preferably, said hydrofluorocarbon is a hydrofluoroalkane selected from the group consisting of pentafluoroethane, 1,1,1,2-tetrafluoroethane, 1,1,2,2-tetrafluoroethane, 1,1-difluoroethane, 1,2-difluoroethane and 1,1,2-trifluoroethane.

Preferably, said membrane M4 has a selectivity of greater than 10, advantageously of greater than 15, preferably of greater than 20, more preferentially of greater than 25, in particular of greater than 30, said selectivity being calculated by the ratio of the permeability of the trifluoroethylene to the permeability of said hydrofluoroalkane through said membrane M4. According to a particularly preferred embodiment, said hydrofluoroalkane is selected from the group consisting of pentafluoroethane, 1,1,1,2-tetrafluoroethane and 1,1,2,2-tetrafluoroethane, and said membrane M4 is made of a material selected from the group consisting of polypropylene, polymethylpentene, cellulose acetate, polyimide, poly[oxy(2,6-dimethyl-1,4-phenylene)] and poly(phenylene oxide).

According to a preferred embodiment which is particularly preferred, said hydrofluorocarbon is a hydrofluoroalkane is selected from the group consisting of pentafluoroethane, 1,1,1,2-tetrafluoroethane and 1,1,2,2-tetrafluoroethane and said membrane M4 is made of a material selected from the group consisting of polypropylene and of polymethylpentene, preferably of polymethylpentene.

According to this fifth aspect, the present invention also provides a process for the production of trifluoroethylene comprising a stage A1) of dehydrofluorination of 1,1,1,2-tetrafluoroethane or a stage of reaction between chlorodifluoromethane and chlorofluoromethane to form a stream comprising trifluoroethylene and 1,1,1,2-tetrafluoroethane and a stage B1) of separation of a stream comprising trifluoroethylene and a hydrofluorocarbon according to the fifth aspect of the present invention with a membrane M4 to form a stream F9′ comprising trifluoroethylene and a stream F10′ comprising said hydrofluorocarbon.

According to a preferred embodiment, said hydrofluorocarbon is a hydrofluoroalkane selected from the group consisting of pentafluoroethane, 1,1,1,2-tetrafluoroethane and 1,1,2,2-tetrafluoroethane, and said membrane M4 is made of a material selected from the group consisting of polypropylene, polymethylpentene, cellulose acetate, polyimide, poly[oxy(2,6-dimethyl-1,4-phenylene)] and poly(phenylene oxide).

According to a preferred embodiment, said hydrofluorocarbon is 1,1,1,2-tetrafluoroethane and said membrane M4 is made of a material selected from the group consisting of polypropylene, polymethylpentene, cellulose acetate, polyimide, poly[oxy(2,6-dimethyl-1,4-phenylene)] and poly(phenylene oxide).

### DETAILED DESCRIPTION OF THE INVENTION

Separation of Hydrogen from a Fluorocarbon

According to a first aspect, the present invention relates to a process for the purification of a fluorocarbon from a mixture comprising said fluorocarbon and hydrogen. Said process comprises a stage (a) of bringing said mixture into contact with a membrane M1 to form a stream F1 comprising the fluorocarbon and a stream F2 comprising the hydrogen.

Preferably, said mixture comprises a molar content of H₂ of less than 50%, preferably of less than 25%, based on the total molar amount of the mixture. Preferably, said mixture comprises a molar content of H₂ of greater than 1%, preferably of greater than 5%, in particular of greater than 10%, based on the total molar amount of the mixture.

Preferably, said mixture is in gaseous form.

The present process thus makes it possible to produce a stream F1 enriched in fluorocarbon, with respect to the initial mixture before being brought into contact with the membrane. Preferably, said stream F1 has a reduced molar content of hydrogen with respect to said mixture. According to a preferred embodiment, said stream F1 comprises at least 25% by weight of fluorocarbon, advantageously at least 30% by weight of fluorocarbon, preferably at least 35% by weight of fluorocarbon, more preferentially at least 40% by weight of fluorocarbon, in particular at least 45% by weight of fluorocarbon, more particularly at least 50% by weight of fluorocarbon, based on the total weight of said stream F1.

Preferably, said stream F1 comprises less than 20% by weight of hydrogen, based on the total weight of said stream F1. Advantageously, said stream F1 comprises less than 15% by weight of hydrogen, preferably less than 10% by weight, in particular less than 5% by weight, more particularly less than 1% by weight, based on the total weight of said stream F1.

In the present process, the stream F2 is enriched in hydrogen. According to a preferred embodiment, said stream F2 has an increased molar content of hydrogen, with respect to said mixture. Preferably, said stream F2 comprises at least 25% by weight of hydrogen, more preferentially at least 50% by weight of hydrogen, in particular at least 75% by weight of hydrogen, more particularly at least 80% by weight of hydrogen, favorably at least 95% by weight of hydrogen, based on the total weight of said stream F2.

Fluorocarbon refers to a compound comprising at least one fluorine atom and at least one carbon atom. The fluorocarbon can, for example, be a hydrofluoroalkane, hydrofluoroolefin, hydrochlorofluoroalkane or hydrochlorofluoroolefin. The term hydrofluoroalkane refers to an alkane compound comprising, as substituents of the carbon atoms, hydrogen atoms and one or more fluorine atom(s). The term hydrofluoroolefin refers to an olefin comprising at least one carbon-carbon double bond and, as substituents of the carbon atoms, hydrogen atoms and one or more fluorine atom(s). The term hydrochlorofluoroalkane refers to an alkane compound comprising, as substituents of the carbon atoms, hydrogen atoms, one or more chlorine atom(s) and one or more fluorine atom(s). The term hydrochlorofluoroolefin refers to an olefin comprising at least one carbon-carbon double bond and, as substituents of the carbon atoms, hydrogen atoms, one or more chlorine atom(s) and one or more fluorine atom(s).

Preferably, said fluorocarbon comprises one, two, three or four carbon atom(s).

Preferably, said fluorocarbon is selected from the group consisting of fluoromethane, difluoromethane, trifluoromethane, trifluoroethylene, 1,1-difluoroethylene, 1,2-difluoroethylene, tetrafluoroethylene, chlorotrifluoroethylene, fluoroethane, pentafluoroethane, 1,1,1,2-tetrafluoroethane, 1,1,2,2-tetrafluoroethane, 1,1-difluoroethane, 1,2-difluoroethane, 1,1,2-trifluoroethane, 1,1,1-trifluoroethane, 2-chloro-1,1,2-trifluoroethane, 1-chloro-1,1,2-trifluoroethane, 2-chloro-1,1,1-trifluoroethane, 3,3,3-trifluoropropene, hexafluoropropene, 1,1,1,3,3,3-hexafluoropropane, 1,1,2,2,3,3-hexafluoropropane, 1,1,1,2,2,3-hexafluoropropane, 1,1,1,2,3,3-hexafluoropropane, 1,1,2,2,3-pentafluoropropane, 1,1,1,2,2-pentafluoropropane, 1,1,2,3,3-pentafluoropropane, 1,1,1,2,3-pentafluoropropane, 1,1,1,3,3-pentafluoropropane, 2-chloro-1,1,1,2-tetrafluoropropane, 3-chloro-1,1,1,3-tetrafluoropropane, 2,3-dichloro-1,1,1-trifluoropropane, 1,1,3,3,3-pentafluoropropene, 1,1,2,3,3-pentafluoropropene, 1,2,3,3,3-pentafluoropropene, 2-chloro-3,3,3-trifluoropropene, 1-chloro-3,3,3-trifluoropropene, 2,3,3,3-tetrafluoropropene, 1,3,3,3-tetrafluoropropene, 1,1,2,3-tetrafluoropropene, 1,1,3,3-tetrafluoropropene, 1,2,3,3-tetrafluoropropene, 1,1,3-trifluoropropene, 1,1,2-trifluoropropene, 3,3,3-trifluoropropene, 1,2,3-trifluoropropene, 2,3,3-trifluoropropene, 1,3,3-trifluoropropene, 1,1-difluoropropene, 1,2-difluoropropene, 2,3-difluoropropene and 3,3-difluoropropene.

In particular, the fluorocarbon is selected from the group consisting of trifluoroethylene, 1,1-difluoroethylene, 1,2-difluoroethylene, tetrafluoroethylene, chlorotrifluoroethylene, pentafluoroethane, 1,1,1,2-tetrafluoroethane, 1,1,2,2-tetrafluoroethane, 1,1-difluoroethane, 1,2-difluoroethane, 1,1,2-trifluoroethane, 2-chloro-1,1,2-trifluoroethane, 1-chloro-1,1,2-trifluoroethane, 2-chloro-1,1,1-trifluoroethane, difluoromethane, trifluoromethane, 2,3,3,3-tetrafluoropropene, 1,3,3,3-tetrafluoropropene, 1-chloro-3,3,3-trifluoropropene, 2-chloro-3,3,3-trifluoropropene, 1,1,1,2,2-pentafluoropropane, 1,1,1,2,3-pentafluoropropane, 1,1,1,3,3-pentafluoropropane, 3-chloro-1,1,1,3-tetrafluoropropane, 2-chloro-1,1,1,2-tetrafluoropropane, 2,3-dichloro-1,1,1-trifluoropropane, hexafluoropropene, 1,2,3,3,3-pentafluoropropene, 3,3,3-trifluoropropene and 1,1,1,2,3,3-hexafluoropropane.

More particularly, the fluorocarbon is selected from the group consisting of trifluoroethylene, 1,1-difluoroethylene, 1,2-difluoroethylene, tetrafluoroethylene, chlorotrifluoroethylene, pentafluoroethane, 1,1,1,2-tetrafluoroethane, 1,1,2,2-tetrafluoroethane, 1,1-difluoroethane, 1,2-difluoroethane, 1,1,2-trifluoroethane, 2-chloro-1,1,2-trifluoroethane, 1-chloro-1,1,2-trifluoroethane, 2-chloro-1,1,1-trifluoroethane, difluoromethane and trifluoromethane.

In the present patent application, the term membrane refers to a membrane which is selectively permeable to one or more compounds so that it makes it possible for the different compounds to migrate through the membrane at different flow rates. The membrane restricts the movement of the molecules which pass through it so that some molecules move more slowly than others or are completely excluded (that is to say, impermeable). For example, the membrane can be selectively permeable to fluorocarbon and impermeable (or weakly permeable) to hydrogen.

The permeability of a membrane depends on its ability to limit or not to limit the diffusion of these compounds through the membrane. The membranes can selectively separate the components over a wide range of solubility parameters and molecular sizes, from macromolecular materials to simple ionic or covalent compounds. The determining property for the performance qualities of the membrane is mainly the selectivity. The membrane separation process is characterized in that a feed stream is divided into two streams: retentate and permeate. The retentate is the portion of the feed which does not pass (or only slightly passes) through the membrane, while the permeate is the portion of the feedstock which passes through the membrane.

In the present patent application, the retentate can be one of the streams described as a function of the membrane used and of the compounds under consideration.

Unlike distillation processes, membrane separation does not require phase separation, which generally makes possible significant savings in energy, compared with distillation processes. The capital costs can also be reduced because membrane separation processes generally have no moving parts, no complex control schemes and few items of auxiliary equipment, compared with other separation processes known in the art.

The membranes can be produced with an extremely high selectivity for the components to be separated. In general, the values of the selectivity are much higher than the typical values of relative volatility for distillation operations. Membrane separation processes may also be able to recover minor but valuable components from the main stream without substantial energy cost. Membrane separation processes are potentially better for the environment since the membrane approach requires the use of relatively simple and nonharmful materials.

According to a preferred embodiment, said membrane M1 is made of a material selected from the group consisting of polyolefin, polyether, polyimide, polyaramid, polyamide, polysulfone, polyvinylidene fluoride, cellulose, polymethyl methacrylate, polytetrafluoroethylene, polyvinyl fluoride, polychlorotrifluoroethylene, polyethylene-tetrafluoroethylene and tetrafluoroethylene/perfluorovinyl ether copolymer optionally substituted by an SO₃H group. Preferably, said membrane M1 is made of a material selected from the group consisting of polyolefin, polyether, polyimide, polymethyl methacrylate, cellulose and polyvinylidene fluoride.

In the present patent application, the term polyolefin refers in particular to polyethylene, polypropylene, polymethylpropene, polybutene, polypentene, polymethylpentene, polymethylbutene, polyhexene, polymethylpentene and polyethylbutene.

In the present patent application, the term polyether refers in particular to a polyaryl ether comprising the monomeric unit —[—O—Ar—]— or —[—Ar¹—O—Ar²—]— in which Ar, Ar¹ and Ar² are, independently of one another, an aromatic ring comprising from 6 to 12 carbon atoms optionally substituted by one or more C₁-C₁₀ alkyl functional groups; preferably, Ar is a phenyl group optionally substituted by one, two, three or four C₁-C₃ alkyl functional groups. In particular, the polyether is poly[oxy(2,6-dimethyl-1,4-phenylene)] or poly(phenylene oxide).

In the present patent application, the cellulose is preferably cellulose acetate.

It can be considered that there is separation between the hydrogen and said fluorocarbon when the selectivity is greater than 2. The higher the selectivity, the more efficient the separation. The process is particularly efficient when the selectivity is greater than 5, preferably greater than 9, in particular greater than 20.

When the permeability of said membrane M1 with regard to the hydrogen is greater than the permeability of said membrane M1 with regard to said fluorocarbon, the selectivity is calculated by the ratio of the permeability of the hydrogen to the permeability of said fluorocarbon under consideration through said membrane M1, i.e. selectivity=[permeability of the hydrogen]/[permeability of said fluorocarbon]. Alternatively, when the permeability of said membrane M1 with regard to the fluorocarbon is greater than the permeability of said membrane with regard to the hydrogen, the selectivity is calculated by the ratio of the permeability of the fluorocarbon under consideration to the permeability of the hydrogen through said membrane, i.e. selectivity=[permeability of said fluorocarbon]/[permeability of the hydrogen].

Preferably, said membrane M1 has a selectivity of greater than 4, advantageously of greater than 5, preferably of greater than 6, more preferentially of greater than 7, in particular of greater than 8, more particularly of greater than 9, said selectivity being calculated by the ratio of the permeability of the hydrogen to the permeability of said fluorocarbon through the membrane. In particular, the selectivity of said membrane M1 can be greater than 10, or greater than 12, or greater than 14, or greater than 16, or greater than 18, or greater than 20, or greater than 22, or greater than 24, or greater than 26, or greater than 28, or greater than 30, or greater than 32, or greater than 34, or greater than 36, or greater than 38, or greater than 40, said selectivity being calculated by the ratio of the permeability of the hydrogen to the permeability of said fluorocarbon through said membrane M1.

Alternatively, said membrane M1 has a selectivity of greater than 4, advantageously of greater than 5, preferably of greater than 6, more preferentially of greater than 7, in particular of greater than 8, more particularly of greater than 9, said selectivity being calculated by the ratio of the permeability of said fluorocarbon to the permeability of the hydrogen through said membrane M1. In particular, the selectivity of said membrane M1 can be greater than 10, or greater than 12, or greater than 14, or greater than 16, or greater than 18, or greater than 20, or greater than 22, or greater than 24, or greater than 26, or greater than 28, or greater than 30, or greater than 32, or greater than 34, or greater than 36, or greater than 38, or greater than 40, said selectivity being calculated by the ratio of the permeability of said fluorocarbon to the permeability of the hydrogen through said membrane M1.

Stage (a) can be carried out over a wide temperature and pressure range.

Preferably, stage (a) of bringing said mixture into contact with said membrane M1 is carried out at a pressure of 0.1 bara to 30 bara, advantageously of 0.2 bara to 25 bara, preferably of 0.3 bara to 20 bara, more preferentially of 0.4 bara to 15 bara, in particular of 0.5 bara to 10 bara, more particularly of 0.5 bara to 5 bara.

Preferably, stage (a) of bringing said mixture into contact with said membrane M1 is carried out at a temperature of 0° C. to 150° C., advantageously of 0° C. to 125° C., preferably of 5° C. to 100° C., more preferentially of 10° C. to 75° C., in particular of 10° C. to 50° C.

During the implementation of the process, a pressure difference is observed between the inlet of the membrane and the outlet of the membrane. The differential pressure expressed here corresponds to the pressure difference existing between the inlet and the outlet of said membrane. Preferably, the differential pressure is from 1 to 3000 kPa, preferably from 50 to 2000 kPa, in particular from 100 to 1000 kPa, more particularly from 100 to 500 kPa.

According to a particular embodiment, said fluorocarbon is trifluoroethylene. Preferably, when said fluorocarbon is trifluoroethylene, said membrane M1 is made of a material selected from the group consisting of polyolefin, polyether, polyvinylidene fluoride, cellulose and polyimide; in particular, said membrane M1 is made of a material selected from the group consisting of polypropylene, polymethylpentene, poly[oxy(2,6-dimethyl-1,4-phenylene)], poly(phenylene oxide), polyvinylidene fluoride, cellulose and polyimide.

According to another particular embodiment, said fluorocarbon is 2,3,3,3-tetrafluoropropene. Preferably, when said fluorocarbon is 2,3,3,3-tetrafluoropropene, said membrane M1 is made of a material selected from the group consisting of polyolefin, polyether, polyvinylidene fluoride, cellulose and polyimide; in particular, said membrane M1 is made of a material selected from the group consisting of polypropylene, polymethylpentene, poly[oxy(2,6-dimethyl-1,4-phenylene)], poly(phenylene oxide), polyvinylidene fluoride, cellulose acetate and polyimide. According to another particular embodiment, said fluorocarbon is pentafluoroethane. Preferably, when said fluorocarbon is pentafluoroethane, said membrane M1 is made of a material selected from the group consisting of polyolefin, polyether, polyvinylidene fluoride, cellulose and polyimide; in particular, said membrane M1 is made of a material selected from the group consisting of polypropylene, polymethylpentene, poly[oxy(2,6-dimethyl-1,4-phenylene)], poly(phenylene oxide), polyvinylidene fluoride, cellulose acetate and polyimide. According to another particular embodiment, said fluorocarbon is hexafluoropropene. Preferably, when said fluorocarbon is hexafluoropropene, said membrane M1 is made of a material selected from the group consisting of polyolefin, polyether, polyvinylidene fluoride, cellulose and polyimide; in particular, said membrane M1 is made of a material selected from the group consisting of polypropylene, polymethylpentene, poly[oxy(2,6-dimethyl-1,4-phenylene)], poly(phenylene oxide), polyvinylidene fluoride, cellulose acetate and polyimide. According to another particular embodiment, said fluorocarbon is 1,1,1,2,3-pentafluoropropene. Preferably, when said fluorocarbon is 1,1,1,2,3-pentafluoropropene, said membrane M1 is made of a material selected from the group consisting of polyolefin, polyether, polyvinylidene fluoride, cellulose and polyimide; in particular, said membrane M1 is made of a material selected from the group consisting of polypropylene, polymethylpentene, poly[oxy(2,6-dimethyl-1,4-phenylene)], poly(phenylene oxide), polyvinylidene fluoride, cellulose acetate and polyimide.

According to another particular embodiment, said fluorocarbon is 1,1-difluoroethylene. Preferably, when said fluorocarbon is 1,1-difluoroethylene, said membrane M1 is made of a material selected from the group consisting of polyolefin, polyether, polyvinylidene fluoride, cellulose and polyimide; in particular, said membrane M1 is made of a material selected from the group consisting of polypropylene, polymethylpentene, poly[oxy(2,6-dimethyl-1,4-phenylene)], poly(phenylene oxide), polyvinylidene fluoride, cellulose acetate and polyimide. According to another particular embodiment, said fluorocarbon is 1,2-difluoroethylene (E and/or Z). Preferably, when said fluorocarbon is 1,2-difluoroethylene (E and/or Z), said membrane M1 is made of a material selected from the group consisting of polyolefin, polyether, polyvinylidene fluoride, cellulose and polyimide; in particular, said membrane M1 is made of a material selected from the group consisting of polypropylene, polymethylpentene, poly[oxy(2,6-dimethyl-1,4-phenylene)], poly(phenylene oxide), polyvinylidene fluoride, cellulose acetate and polyimide.

According to another particular embodiment, said fluorocarbon is chlorotrifluoroethylene. Preferably, when said fluorocarbon is chlorotrifluoroethylene, said membrane M1 is made of a material selected from the group consisting of polyolefin, polyether, polyvinylidene fluoride, cellulose and polyimide; in particular, said membrane M1 is made of a material selected from the group consisting of polypropylene, polymethylpentene, poly[oxy(2,6-dimethyl-1,4-phenylene)], poly(phenylene oxide), polyvinylidene fluoride, cellulose acetate and polyimide.

According to a preferred embodiment, in the present process, the hydrogen is preferably in anhydrous form. According to a preferred embodiment, the fluorocarbon is preferably in anhydrous form. The term anhydrous refers to a content by weight of water of less than 1000 ppm, advantageously 500 ppm, preferably of less than 200 ppm, in particular of less than 100 ppm, on the basis of the total weight of the compound under consideration.

Said mixture used in this process and brought into contact with said membrane M1 can also contain nitrogen. When this mixture is subjected to stage (a) of the present process, said stream F1 also comprises nitrogen. Said stream F1 can be subjected to a second membrane purification stage. Said process comprises a stage (b) of bringing said stream F1 into contact with a membrane M1′ to form a stream F3 comprising said fluorocarbon and a stream F4 comprising nitrogen.

According to a first embodiment, said membrane M1′ can be more permeable to said fluorocarbon than to the nitrogen. Thus, said membrane M1′ has a selectivity of greater than 2, advantageously of greater than 3, preferably of greater than 4, more preferentially of greater than 5, in particular of greater than 6, more particularly of greater than 7, said selectivity being calculated by the ratio of the permeability of said fluorocarbon to the permeability of the nitrogen through said membrane M1′.

Preferably, in this embodiment, said fluorocarbon is a hydrofluoroolefin or a hydrochlorofluoroolefin. In particular, said fluorocarbon is a hydrofluoroolefin. More particularly, stage b) can be carried out under the conditions described below in embodiment 1 according to the process for the separation of nitrogen from a fluorocarbon.

Preferably, in this embodiment, said membrane M1′ is made of polyolefin, polyether or contains a siloxane functional group. Preferably, said second membrane is made of polypropylene, polymethylpentene or polyalkylsiloxane. The polyalkylsiloxane is preferably polydimethylsiloxane.

Alternatively, according to a second embodiment, said membrane M1′ is more permeable to the nitrogen than to the fluorocarbon. Thus, said membrane M1′ has a selectivity of greater than 2, advantageously of greater than 3, preferably of greater than 4, more preferentially of greater than 5, in particular of greater than 6, more particularly of greater than 7, said selectivity being calculated by the ratio of the permeability of the nitrogen to the permeability of said fluorocarbon through said membrane M1′. In particular, the selectivity of said membrane M1′ can be greater than 10, or greater than 12, or greater than 14, or greater than 16, or greater than 18, or greater than 20, or greater than 22, or greater than 24, or greater than 26, or greater than 28, or greater than 30, or greater than 32, or greater than 34, or greater than 36, or greater than 38, or greater than 40, said selectivity being calculated by the ratio of the permeability of said fluorocarbon to the permeability of the nitrogen through said membrane M1′. Preferably, in this embodiment, said fluorocarbon is a hydrofluoroalkane or a hydrochlorofluoroalkane, in particular a hydrofluoroalkane. More particularly, stage b) can be carried out under the conditions described below in embodiment 2 according to the process for the separation of nitrogen from a fluorocarbon. In particular, in this embodiment, said membrane M1′ is made of polyolefin. More particularly, said membrane M1′ is made of a material selected from the group consisting of polyethylene, polypropylene, polymethylpropene, polybutene, polypentene, polymethylpentene, polymethylbutene, polyhexene, polymethylpentene and polyethylbutene. Favorably, said membrane M1′ is made of a material selected from the group consisting of polypropylene and of polymethylpentene.

Alternatively, according to a third embodiment, when said mixture contains nitrogen, hydrogen and the fluorocarbon, stage (a) of the present process makes it possible to simultaneously separate the nitrogen and the hydrogen from the fluorocarbon. Thus, in this embodiment, the present process comprises a stage (a) of bringing said mixture into contact with a membrane M1″ to form a stream F1′ comprising the fluorocarbon and a stream F2′ comprising the hydrogen and nitrogen. Said membrane M1″ can have a selectivity of greater than 5, advantageously of greater than 10, preferably of greater than 15, more preferentially of greater than 20, in particular of greater than 25, more particularly of greater than 30, when the selectivity is calculated by the ratio of the permeability of said fluorocarbon to the permeability of the nitrogen through said membrane M1″, and said first membrane can have a selectivity of greater than 5, advantageously of greater than 10, preferably of greater than 15, more preferentially of greater than 20, in particular of greater than 25, more particularly of greater than 30, when the selectivity is calculated by the ratio of the permeability of said fluorocarbon to the permeability of the hydrogen through said membrane M1″. Alternatively, said membrane M1″ can have a selectivity of greater than 5, advantageously of greater than 10, preferably of greater than 15, more preferentially of greater than 20, in particular of greater than 25, more particularly of greater than 30, when the selectivity is calculated by the ratio of the permeability of the nitrogen to the permeability of said fluorocarbon through said membrane M1″, and said membrane M1″ can have a selectivity of greater than 5, advantageously of greater than 10, preferably of greater than 15, more preferentially of greater than 20, in particular of greater than 25, more particularly of greater than 30, when the selectivity is calculated by the ratio of the permeability of the hydrogen to the permeability of said fluorocarbon through said membrane M1″.

Alternatively, according to a fourth embodiment, when said mixture contains nitrogen, hydrogen and the fluorocarbon, the nitrogen can be separated from the fluorocarbon and from the hydrogen before stage (a) of the present process. In this case, the present process comprises a stage of bringing said mixture into contact with a membrane M1′ to form a stream F1″ comprising the fluorocarbon and the hydrogen and a stream F2″ comprising the nitrogen. Said stream F1″ is subsequently subjected to stage (a) according to the present process, i.e. said stream F1″ is brought into contact with said membrane M1 as defined above according to the first aspect of the invention.

In this case, said membrane M1′ can have a selectivity of greater than 2, advantageously of greater than 3, preferably of greater than 4, more preferentially of greater than 5, in particular of greater than 6, more particularly of greater than 7, when the selectivity is calculated by the ratio of the permeability of said fluorocarbon to the permeability of the nitrogen through the membrane, and said membrane M1′ can have a selectivity of greater than 2, advantageously of greater than 3, preferably of greater than 4, more preferentially of greater than 5, in particular of greater than 6, more particularly of greater than 7, when the selectivity is calculated by the ratio of the permeability of the hydrogen to the permeability of the nitrogen through the membrane. Alternatively, according to this fourth embodiment, said membrane M1′ can have a selectivity of greater than 2, advantageously of greater than 3, preferably of greater than 4, more preferentially of greater than 5, in particular of greater than 6, more particularly of greater than 7, when the selectivity is calculated by the ratio of the permeability of the nitrogen to the permeability of said fluorocarbon through the membrane, and said membrane M1′ can have a selectivity of greater than 2, advantageously of greater than 3, preferably of greater than 4, more preferentially of greater than 5, in particular of greater than 6, more particularly of greater than 7, when the selectivity is calculated by the ratio of the permeability of the nitrogen to the permeability of the hydrogen through the membrane. Thus, in this embodiment, said membrane M1′ can be made of a material as described above in the first or the second embodiment depending on said fluorocarbon under consideration.

In this first aspect of the present invention, said membrane M1, said membrane M1′ and the membrane M1″ are selected, independently of one another, from a film, a laminated structure, hollow fibers and coated fibers. The membrane is selected as a function of its selectivity with regard to the compounds to be separated. The membrane can be provided on an inert support. Hereinbelow, the present patent application describes a process for the separation of nitrogen from a fluorocarbon. The particular embodiments described in this aspect of the invention can be combined with the first aspect of the present invention.

Generally, stage b) is carried out at a pressure of 0.1 bara to 30 bara, advantageously of 0.2 bara to 25 bara, preferably of 0.3 bara to 20 bara, more preferentially of 0.4 bara to 15 bara, in particular of 0.5 bara to 10 bara, more particularly of 0.5 bara to 5 bara. Preferably, stage b) is carried out at a temperature of 0° C. to 150° C., advantageously of 0° C. to 125° C., preferably of 5° C. to 100° C., more preferentially of 10° C. to 75° C., in particular of 10° C. to 50° C. During the implementation of this stage, a pressure difference is observed between the inlet of the membrane and the outlet of the membrane. The differential pressure expressed here corresponds to the pressure difference existing between the inlet and the outlet of said membrane. Preferably, the differential pressure is from 1 to 3000 kPa, preferably from 50 to 2000 kPa, in particular from 100 to 1000 kPa, more particularly from 100 to 500 kPa.

### Separation of the Nitrogen from a Fluorocarbon

### Embodiment 1

According to another aspect of the present invention, a process for the separation of a mixture comprising a hydrofluoroolefin and nitrogen is provided. According to a preferred embodiment, said process comprises a stage of bringing said mixture into contact with a membrane M3 to form a stream F7 comprising said one hydrofluoroolefin and a stream F8 comprising the nitrogen. Preferably, said membrane M3 is made of a material containing a siloxane functional group.

Preferably, said membrane M3 is made of a material containing a functional group of formula —[—(R)(R′)Si—O]ₙ— with R and R′ independently selected from the group consisting of hydrogen, C₁-C₂₀ alkyl, C₃-C₁₀ cycloalkyl, C₆-C₁₂ aryl and n being an integer greater than 50, preferably greater than 100, in particular greater than 1000.

Preferably, said membrane M3 is made of polyalkylsiloxane. According to the present invention, in the polyalkylsiloxane compound, the term alkyl refers to a radical of C₁-C₁₀ alkyl type.

In particular, said membrane M3 is made of polydimethylsiloxane.

According to a preferred embodiment, said membrane M3 is chosen from a film, a laminated structure, hollow fibers and coated fibers.

Preferably, said hydrofluoroolefin is selected from the group consisting of trifluoroethylene, 1,1-difluoroethylene, 1,2-difluoroethylene, tetrafluoroethylene, 3,3,3-trifluoropropene, hexafluoropropene, 1,1,3,3,3-pentafluoropropene, 1,1,2,3,3-pentafluoropropene, 1,2,3,3,3-pentafluoropropene, 2,3,3,3-tetrafluoropropene, 1,3,3,3-tetrafluoropropene, 1,1,2,3-tetrafluoropropene, 1,1,3,3-tetrafluoropropene, 1,2,3,3-tetrafluoropropene, 1,1,3-trifluoropropene, 1,1,2-trifluoropropene, 3,3,3-trifluoropropene, 1,2,3-trifluoropropene, 2,3,3-trifluoropropene, 1,3,3-trifluoropropene, 1,1-difluoropropene, 1,2-difluoropropene, 2,3-difluoropropene and 3,3-difluoropropene.

In particular, said hydrofluoroolefin is selected from the group consisting of trifluoroethylene, 1,1-difluoroethylene, 1,2-difluoroethylene, tetrafluoroethylene, 2,3,3,3-tetrafluoropropene, 1,3,3,3-tetrafluoropropene, 1,1,1,2,2-pentafluoropropane, 1,1,1,2,3-pentafluoropropane, 1,1,1,3,3-pentafluoropropane, hexafluoropropene, 1,2,3,3,3-pentafluoropropene and 3,3,3-trifluoropropene.

Preferably, the process is carried out at a pressure of 0.1 bara to 30 bara, advantageously of 0.2 bara to 25 bara, preferably of 0.3 bara to 20 bara, more preferentially of 0.4 bara to 15 bara, in particular of 0.5 bara to 10 bara, more particularly of 0.5 bara to 5 bara. Preferably, this stage is carried out at a temperature of 0° C. to 150° C., advantageously of 0° C. to 125° C., preferably of 5° C. to 100° C., more preferentially of 10° C. to 75° C., in particular of 10° C. to 50° C. During the implementation of this process, a pressure difference is observed between the inlet of the membrane and the outlet of the membrane. The differential pressure expressed here corresponds to the pressure difference existing between the inlet and the outlet of said membrane. Preferably, the differential pressure is from 1 to 3000 kPa, preferably from 50 to 2000 kPa, in particular from 100 to 1000 kPa, more particularly from 100 to 500 kPa.

Preferably, said membrane M3 has a selectivity of greater than 5, advantageously of greater than 6, preferably of greater than 7, more preferentially of greater than 8, in particular of greater than 9, said selectivity being calculated by the ratio of the permeability of said hydrofluoroolefin to the permeability of the nitrogen through said membrane M3.

According to a particularly preferred embodiment, said hydrofluoroolefin is trifluoroethylene and said membrane M3 is made of polydimethylsiloxane.

According to another particularly preferred embodiment, said hydrofluoroolefin is 2,3,3,3-tetrafluoropropene and said membrane M3 is made of polydimethylsiloxane.

According to another particularly preferred embodiment, said hydrofluoroolefin is hexafluoropropene and said membrane M3 is made of polydimethylsiloxane.

According to another particularly preferred embodiment, said hydrofluoroolefin is 1,1,1,2,3-pentafluoropropene and said membrane M3 is made of polydimethylsiloxane.

According to another particularly preferred embodiment, said hydrofluoroolefin is 1,1-difluoroethylene and said membrane M3 is made of polydimethylsiloxane.

According to another particularly preferred embodiment, said hydrofluoroolefin is 1,2-difluoroethylene and said membrane M3 is made of polydimethylsiloxane.

In this embodiment, the present process makes it possible to enrich the stream F7 in hydrofluoroolefin, with respect to the starting mixture. The stream F8 is for its part enriched in nitrogen, with respect to the initial mixture.

### Embodiment 2

According to another aspect of the present invention, a process for the separation of a mixture comprising a hydrofluoroalkane and nitrogen is provided.

According to a preferred embodiment, said process comprises a stage of bringing said mixture into contact with a membrane M3′ to form a stream F7′ comprising said hydrofluoroalkane and a stream F8′ comprising the nitrogen.

Preferably, said membrane M3′ is made of polyolefin.

According to a preferred embodiment, said membrane M3′ is chosen from a film, a laminated structure, hollow fibers and coated fibers.

Preferably, said membrane M3′ is made of a material selected from the group consisting of polyethylene, polypropylene, polymethylpropene, polybutene, polypentene, polymethylpentene, polymethylbutene, polyhexene, polymethylpentene and polyethylbutene.

In particular, said membrane is made of polypropylene or of polymethylpentene.

According to a preferred embodiment, said hydrofluoroalkane is selected from the group consisting of pentafluoroethane, 1,1,1,2-tetrafluoroethane, 1,1,2,2-tetrafluoroethane, 1,1-difluoroethane, 1,2-difluoroethane, 1,1,2-trifluoroethane, fluoromethane, difluoromethane, trifluoromethane, 1,1,1,2,2-pentafluoropropane, 1,1,1,2,3-pentafluoropropane, 1,1,1,3,3-pentafluoropropane and 1,1,1,2,3,3-hexafluoropropane.

Preferably, said membrane M3′ has a selectivity of greater than 10, advantageously of greater than 15, preferably of greater than 20, more preferentially of greater than 25, in particular of greater than 30, said selectivity being calculated by the ratio of the permeability of the nitrogen to the permeability of said hydrofluoroalkane through said membrane M3′.

In a particularly preferred embodiment, said hydrofluoroalkane is pentafluoroethane and said membrane M3′ is made of polypropylene or of polymethylpentene, preferably of polymethylpentene.

Preferably, the process is carried out at a pressure of 0.1 bara to 30 bara, advantageously of 0.2 bara to 25 bara, preferably of 0.3 bara to 20 bara, more preferentially of 0.4 bara to 15 bara, in particular of 0.5 bara to 10 bara, more particularly of 0.5 bara to 5 bara. Preferably, this stage is carried out at a temperature of 0° C. to 150° C., advantageously of 0° C. to 125° C., preferably of 5° C. to 100° C., more preferentially of 10° C. to 75° C., in particular of 10° C. to 50° C. During the implementation of this process, a pressure difference is observed between the inlet of the membrane and the outlet of the membrane. The differential pressure expressed here corresponds to the pressure difference existing between the inlet and the outlet of said membrane. Preferably, the differential pressure is from 1 to 3000 kPa, preferably from 50 to 2000 kPa, in particular from 100 to 1000 kPa, more particularly from 100 to 500 kPa.

In this embodiment, the present process makes it possible to enrich the stream F7′ in hydrofluoroalkane, with respect to the starting mixture. The stream F8′ is for its part enriched in nitrogen, with respect to the initial mixture.

Preferably, in the embodiment 1 and the embodiment 2, the nitrogen is in anhydrous form. Preferably, in the embodiment 1 and the embodiment 2, the hydrofluoroolefin and the hydrofluoroalkane are in anhydrous form. The term anhydrous refers to a content by weight of water of less than 1000 ppm, advantageously 500 ppm, preferably of less than 200 ppm, in particular of less than 100 ppm, on the basis of the total weight of the compound under consideration.

### Process for the Production of Trifluoroethylene

The present patent application describes, in the first, second and third aspects of the present invention, a process for the separation of hydrogen and/or of nitrogen from a fluorocarbon. The present process is particularly advantageous in the process for the purification of a fluorocarbon, such as trifluoroethylene. As mentioned above, trifluoroethylene is used as monomers or comonomers in the manufacture of fluorocarbon polymers exhibiting noteworthy characteristics, in particular excellent chemical resistance and good thermal resistance. Trifluoroethylene is also used as refrigerant. For these applications, a high trifluoroethylene purity is sought, while employing an efficient and environmentally friendly process.

Thus, according to a fourth aspect, the present invention provides a process for the production of trifluoroethylene. The present invention provides a process for the production of trifluoroethylene in a reactor furnished with a fixed catalytic bed comprising a catalyst, said process comprising a stage A) of reaction of chlorotrifluoroethylene with hydrogen in the presence of the catalyst and in the gas phase to produce a stream comprising the trifluoroethylene, chlorotrifluoroethylene and unreacted hydrogen and a stage B) of bringing a stream comprising trifluoroethylene, chlorotrifluoroethylene and possibly hydrogen into contact with a membrane M2 to form a stream F5 comprising trifluoroethylene and possibly hydrogen and a stream F6 comprising chlorotrifluoroethylene and possibly hydrogen. Preferably, the stream comprising trifluoroethylene, chlorotrifluoroethylene and hydrogen used in stage B) is said stream comprising the trifluoroethylene, chlorotrifluoroethylene and unreacted hydrogen produced in stage A). The hydrogen can be in the stream F5 or in the stream F6 depending on the membrane M2 used.

Surprisingly, it has been demonstrated by the present patent application that it is possible to separate the trifluoroethylene from the chlorotrifluoroethylene and/or from the hydrogen, both starting materials in the present membrane separation process.

It has been demonstrated that said membrane M2 is particularly effective in removing most of the chlorotrifluoroethylene, one of the unreacted starting reactants in stage A) of the present process. The permeability of the membrane M2 with regard to trifluoroethylene is markedly different from that to chlorotrifluoroethylene and possibly from that to hydrogen. Stage B) thus makes it possible to remove a significant amount of chlorotrifluoroethylene and possibly of hydrogen from the trifluoroethylene stream (stream F5), which will make it possible to facilitate the operations of purification of the latter. For example, trifluoroethylene is generally separated from chlorotrifluoroethylene by cryogenic distillation. By implementing stage B) of the present process, the subsequent distillation will be facilitated and will be able, for example, to be carried out in a plant of reduced dimensions. Stage B) can be carried out directly after stage A) or said stream resulting from stage A) can be treated following stages i), ii) and iii) described below before carrying out stage B). In this case, the stream subjected to stage B) comprises trifluoroethylene and chlorotrifluoroethylene, the hydrogen having been removed by stage iii) described below.

Thus, said stream F5 is enriched in trifluoroethylene, with respect to the stream employed in stage B). Said stream F6 is enriched in chlorotrifluoroethylene, with respect to the stream employed in stage B).

Preferably, said stream F6 comprising chlorotrifluoroethylene and possibly hydrogen is recovered and recycled in stage A). The stream F5 comprising trifluoroethylene can be purified as explained below. Said stage B) makes it possible to remove all or a portion of the chlorotrifluoroethylene and optionally of the hydrogen which has not reacted in stage A). This stage thus makes it possible to limit the amount of unreacted starting materials in the subsequent purification stages, thus facilitating the purification of the trifluoroethylene as explained above.

According to a preferred embodiment, the process is carried out in continuous mode.

According to a preferred embodiment, the hydrogen is in anhydrous form. According to a preferred embodiment, the chlorotrifluoroethylene is in anhydrous form. The implementation of the process in the presence of anhydrous hydrogen and/or chlorotrifluoroethylene makes it possible to effectively increase the lifetime of the catalyst and thus the overall productivity of the process. In the present patent application, the term anhydrous refers to a content by weight of water of less than 1000 ppm, advantageously 500 ppm, preferably of less than 200 ppm, in particular of less than 100 ppm, on the basis of the total weight of the compound under consideration.

Stage A) of the process for the production of trifluoroethylene is carried out in the presence of a catalyst. Preferably, the catalyst is based on a metal from columns 8 to 10 of the Periodic Table of the Elements. In particular, the catalyst is based on a metal selected from the group consisting of Pd, Pt, Rh, and Ru; preferably palladium. Preferably, the catalyst is supported. The support is preferably selected from the group consisting of activated carbon, an aluminum-based support, calcium carbonate and graphite. Preferably, the support is based on aluminum. In particular, the support is alumina. The alumina can be α-alumina. Preferably, the alumina comprises at least 90% of α-alumina. It was observed that the conversion of the hydrogenolysis reaction was improved when the alumina is an α-alumina. Thus, the catalyst is more particularly palladium supported on alumina, advantageously palladium supported on an alumina comprising at least 90% of α-alumina, preferably palladium supported on an α-alumina. Preferably, the palladium represents from 0.01% to 5% by weight, on the basis of the total weight of the catalyst, preferably from 0.1% to 2% by weight, on the basis of the total weight of the catalyst. In particular, said catalyst comprises from 0.01% to 5% by weight of palladium supported on alumina; preferably, the alumina comprises at least 90% of α-alumina; more preferentially, the alumina is an α-alumina.

Said catalyst is preferably activated before its use in stage A). Preferably, the activation of the catalyst is carried out at high temperature and in the presence of a reducing agent. According to a particular embodiment, the reducing agent is chosen from the group consisting of hydrogen, carbon monoxide, nitrogen monoxide, formaldehyde, C₁-C₆ alkanes and C₁-C₁₀ hydrohalocarbons, or a mixture of these; preferably hydrogen and a C₁-C₁₀ hydrohalocarbon, or a mixture of these; in particular hydrogen, chlorotrifluoroethylene, trifluoroethylene, chlorotrifluoroethane, trifluoroethane and difluoroethane, or a mixture of these. Preferably, the activation of the catalyst is carried out at a temperature of between 100° C. and 400° C., in particular at a temperature of between 150° C. and 350° C. In particular, the activation of the catalyst is carried out at a temperature of between 100° C. and 400° C., in particular at a temperature of between 150° C. and 350° C., in the presence of hydrogen as reducing agent.

Said catalyst used in the present process can be regenerated. This regeneration stage can be carried out in a temperature range of the catalytic bed of between 90° C. and 450° C. Preferably, the regeneration stage is carried out in the presence of hydrogen. Carrying out the regeneration stage makes it possible to improve the yield of the reaction compared with the initial yield before regeneration. According to a preferred embodiment, the regeneration stage can be carried out at a temperature of the catalytic bed of 90° C. to 300° C., preferably at a temperature of the catalytic bed of 90° C. to 250° C., more preferentially of 90° C. to 200° C., in particular of 90° C. to 175° C., more particularly at a temperature of the catalytic bed of 90° C. to 150° C. In particular, carrying out the regeneration stage at a low temperature, for example of 90° C. to 200° C. or of 90° C. to 175° C. or of 90° C. to 150° C., makes possible the desorption of compounds which are harmful to the activity of the catalyst and/or makes it possible to limit phase transitions which modify the structure of the catalyst. According to another preferred embodiment, the regeneration stage can be carried out at a temperature of the catalytic bed of greater than 200° C., advantageously of greater than 230° C., preferably of greater than 250° C., in particular of greater than 300° C. The regeneration stage can be carried out periodically as a function of the productivity or of the conversion obtained in stage a). The regeneration stage can be advantageously carried out at a temperature of the catalytic bed of between 200° C. and 300° C., preferably between 205° C. and 295° C., more preferentially between 210° C. and 290° C., in particular between 215° C. and 290° C., more particularly between 220° C. and 285° C., favorably between 225° C. and 280° C., more favorably between 230° C. and 280° C. Alternatively, the regeneration stage can be carried out at a temperature of between 300° C. and 450° C., preferably between 300° C. and 400° C. The regenerated catalyst can be reused in stage A) of the present process.

The process comprises, as mentioned above, a stage of reaction of chlorotrifluoroethylene (CTFE) with hydrogen in order to produce a stream comprising trifluoroethylene. This hydrogenolysis stage is carried out in the presence of a catalyst and in the gas phase. Preferably, the hydrogenolysis stage is carried out in the presence of a preactivated catalyst and in the gas phase. The hydrogenolysis stage consists in simultaneously introducing hydrogen, CTFE and optionally an inert gas, such as nitrogen, in the gas phase and in the presence of said catalyst, which is preferably activated. Preferably, said stage A) is carried out at a temperature of the fixed catalytic bed of between 50° C. and 250° C. Said stage A) can be carried out at a temperature of the fixed catalytic bed of between 50° C. and 240° C., advantageously between 50° C. and 230° C., preferably between 50° C. and 220° C., more preferentially between 50° C. and 210° C., in particular between 50° C. and 200° C. Said stage A) can also be carried out at a temperature of the fixed catalytic bed of between 60° C. and 250° C., advantageously between 70° C. and 250° C., preferably between 80° C. and 250° C., more preferentially between 90° C. and 250° C., in particular between 100° C. and 250° C., more particularly between 120° C. and 250° C. Said stage A) can also be carried out at a temperature of the fixed catalytic bed of between 60° C. and 240° C., advantageously between 70° C. and 230° C., preferably between 80° C. and 220° C., more preferentially between 90° C. and 210° C., in particular between 100° C. and 200° C., more particularly between 100° C. and 180° C., favorably between 100° C. and 160° C., particularly preferably between 120° C. and 160° C. The H₂/CTFE molar ratio is of between 0.5/1 and 2/1 and preferably of between 1/1 and 1.2/1. If an inert gas, such as nitrogen, is present in stage A), the nitrogen/H₂ molar ratio is of between 0/1 and 2/1 and preferably of between 0/1 and 1/1. Stage A) is preferably carried out at a pressure of 0.05 MPa to 1.1 MPa, more preferentially of 0.05 MPa to 0.5 MPa, in particular at atmospheric pressure. The contact time, calculated as being the ratio of the volume, in liters, of catalyst to the total flow rate of the gas mixture, in standard liters per second, at the inlet of the reactor, is of between 1 and 60 seconds, preferably between 5 and 45 seconds, in particular between 10 and 30 seconds, more particularly between 15 and 25 seconds. The hydrogenolysis stage (stage A)) of the present process results in the production of a stream comprising trifluoroethylene. Said stream can also contain unreacted chlorotrifluoroethylene and hydrogen. Said stream can also contain nitrogen. Said stream can also comprise HCl or HF or a mixture of the two. Said stream can also comprise organic impurities (such as F143, F133 and other organics).

Stage B) results in the formation of a stream F5 comprising trifluoroethylene and possibly hydrogen and of a stream F6 comprising chlorotrifluoroethylene and possibly hydrogen.

Preferably, said membrane M2 is made of a material selected from the group consisting of polyolefin, polyether, polyimide, polyaramid, polyamide, polysulfone, polyvinylidene fluoride, cellulose, polymethyl methacrylate, polytetrafluoroethylene, polyvinyl fluoride, polychlorotrifluoroethylene, polyethylene-tetrafluoroethylene and tetrafluoroethylene/perfluorovinyl ether copolymer optionally substituted by an SO₃H group. Preferably, said membrane M2 is made of a material selected from the group consisting of polyolefin, polyether, polyimide, polymethyl methacrylate, cellulose and polyvinylidene fluoride. The terms polyolefin and polyether are defined above in connection with the first aspect of the present invention. Preferably, the cellulose is cellulose acetate.

In particular, said membrane M2 is made of a material selected from the group consisting of polypropylene, polymethylpentene, poly[oxy(2,6-dimethyl-1,4-phenylene)], poly(phenylene oxide), cellulose acetate, polyvinylidene fluoride and polyimide.

Preferably, stage B) is carried out at a pressure of 0.1 bara to 30 bara, advantageously of 0.2 bara to 25 bara, preferably of 0.3 bara to 20 bara, more preferentially of 0.4 bara to 15 bara, in particular of 0.5 bara to 10 bara, more particularly of 0.5 bara to 5 bara. During the implementation of stage B), a pressure difference is observed between the inlet of the membrane and the outlet of the membrane. The differential pressure expressed here corresponds to the pressure difference existing between the inlet and the outlet of said membrane. Preferably, the differential pressure is from 1 to 3000 kPa, preferably from 50 to 2000 kPa, in particular from 100 to 1000 kPa, more particularly from 100 to 500 kPa. It was observed that the differential pressure could influence the value of the permeability of the chlorotrifluoroethylene. Thus, to obtain a selectivity of greater than 5, when the selectivity is calculated by the ratio of the permeability of the trifluoroethylene to the permeability of the chlorotrifluoroethylene, a differential pressure is of approximately 2.5 bar. To obtain a selectivity of greater than 10, when the selectivity is calculated by the ratio of the permeability of the trifluoroethylene to the permeability of the chlorotrifluoroethylene, a differential pressure is of approximately 3.5 bar.

Preferably, stage B) is carried out at a temperature of 0° C. to 150° C., advantageously of 0° C. to 125° C., preferably of 5° C. to 100° C., more preferentially of 10° C. to 75° C., in particular of 10° C. to 50° C.

Preferably, said membrane M2 has a selectivity of greater than 5, advantageously of greater than 6, preferably of greater than 7, more preferentially of greater than 8, in particular of greater than 9, when the selectivity is calculated by the ratio of the permeability of the hydrogen to the permeability of the trifluoroethylene through said membrane M2.

Preferably, said membrane M2 has a selectivity of greater than 5, advantageously of greater than 6, preferably of greater than 7, more preferentially of greater than 8, in particular of greater than 9, when the selectivity is calculated by the ratio of the permeability of the chlorotrifluoroethylene to the permeability of the trifluoroethylene through said membrane M2.

In particular, said membrane M2 has a selectivity of greater than 10, or of greater than 12, or of greater than 14, or of greater than 16, or of greater than 18, or of greater than 20, or of greater than 22, or of greater than 24, when the selectivity is calculated by the ratio of the permeability of the chlorotrifluoroethylene to the permeability of the trifluoroethylene through said membrane M2.

Thus, in order to efficiently separate the chlorotrifluoroethylene and the hydrogen from the trifluoroethylene, said membrane M2 has a selectivity of greater than 5, advantageously of greater than 6, preferably of greater than 7, more preferentially of greater than 8, in particular of greater than 9, when the selectivity is calculated by the ratio of the permeability of the hydrogen to the permeability of the trifluoroethylene through the membrane, and said membrane M2 has a selectivity of greater than 5, advantageously of greater than 6, preferably of greater than 7, more preferentially of greater than 8, in particular of greater than 9, when the selectivity is calculated by the ratio of the permeability of the chlorotrifluoroethylene to the permeability of the trifluoroethylene through the membrane.

In particular, said membrane M2 has a selectivity of greater than 5, advantageously of greater than 6, preferably of greater than 7, more preferentially of greater than 8, in particular of greater than 9, when the selectivity is calculated by the ratio of the permeability of the hydrogen to the permeability of the trifluoroethylene through the membrane, and said membrane M2 has a selectivity of greater than 10, or of greater than 12, or of greater than 14, or of greater than 16, or of greater than 18, or of greater than 20, or of greater than 22, or of greater than 24, when the selectivity is calculated by the ratio of the permeability of the chlorotrifluoroethylene to the permeability of the trifluoroethylene through the membrane.

Thus, said membrane M2 is more permeable to the hydrogen and to the chlorotrifluoroethylene than to the trifluoroethylene, which makes possible an advantageous separation of the stream resulting from stage A). This embodiment, with the selectivities mentioned above, is preferably obtained when said membrane M2 is made of a material consisting of polyolefin, in particular polypropylene or polymethylpentene.

Alternatively, said membrane M2 is more permeable to the hydrogen and to the trifluoroethylene than to the chlorotrifluoroethylene, which makes possible an advantageous separation of the stream resulting from stage A). This embodiment, with the selectivities mentioned below, is preferably obtained when said membrane M2 is made of a material consisting of polyimide or of cellulose, in particular of polyimide or of cellulose acetate.

Preferably, said membrane M2 has a selectivity of greater than 10, advantageously of greater than 20, preferably of greater than 50, more preferentially of greater than 75, in particular of greater than 100, when the selectivity is calculated by the ratio of the permeability of the hydrogen to the permeability of the chlorotrifluoroethylene through said membrane M2. Preferably, said membrane M2 has a selectivity of greater than 5, advantageously of greater than 6, preferably of greater than 7, more preferentially of greater than 8, in particular of greater than 9, when the selectivity is calculated by the ratio of the permeability of the trifluoroethylene to the permeability of the chlorotrifluoroethylene through said membrane M2.

In particular, said membrane M2 has a selectivity of greater than 10, or of greater than 12, or of greater than 14, when the selectivity is calculated by the ratio of the permeability of the trifluoroethylene to the permeability of the chlorotrifluoroethylene through said membrane M2.

Thus, in order to efficiently separate the chlorotrifluoroethylene from the hydrogen and from the trifluoroethylene, said membrane M2 has a selectivity of greater than 10, advantageously of greater than 20, preferably of greater than 50, more preferentially of greater than 75, in particular of greater than 100, when the selectivity is calculated by the ratio of the permeability of the hydrogen to the permeability of the chlorotrifluoroethylene through the membrane, and said membrane M2 has a selectivity of greater than 5, advantageously of greater than 6, preferably of greater than 7, more preferentially of greater than 8, in particular of greater than 9, when the selectivity is calculated by the ratio of the permeability of the trifluoroethylene to the permeability of the chlorotrifluoroethylene through the membrane.

In particular, said membrane M2 has a selectivity of greater than 10, advantageously of greater than 20, preferably of greater than 50, more preferentially of greater than 75, in particular of greater than 100, when the selectivity is calculated by the ratio of the permeability of the hydrogen to the permeability of the chlorotrifluoroethylene through the membrane, and said membrane M2 has a selectivity of greater than 10, or of greater than 12, or of greater than 14, when the selectivity is calculated by the ratio of the permeability of the trifluoroethylene to the permeability of the chlorotrifluoroethylene through the membrane.

According to a preferred embodiment, said stream F5 comprises at least 25% by weight of trifluoroethylene, advantageously at least 30% by weight, preferably at least 35% by weight, more preferentially at least 40% by weight, in particular at least 45% by weight, more particularly at least 50% by weight, of trifluoroethylene, based on the total weight of said stream F5. According to a particularly preferred embodiment, said stream F5 comprises at least 55% by weight of trifluoroethylene, advantageously at least 60% by weight, preferably at least 70% by weight, more preferentially at least 80% by weight, in particular at least 90% by weight, more particularly at least 95% by weight, of trifluoroethylene, based on the total weight of said stream F5.

Said stream F5 can comprise a small amount of chlorotrifluoroethylene. Preferably, said stream F5 comprises a content by weight of chlorotrifluoroethylene of less than 40%, preferably of less than 30%, more preferentially of less than 20%, in particular of less than 10%, more particularly of less than 5%, based on the total weight of said stream F5.

The present process can comprise additional stages i) to iv). These stages can be carried out starting from the stream F5 or starting from said stream resulting from stage A). As explained above, stage B) can be carried out starting from said stream resulting from stage A) or starting from said stream resulting from stage A) pretreated by stages i), ii) and optionally iii) to remove certain products.

Said process can comprise the stages of:
i) removal of HF and/or HCl to form a gas mixture;
ii) drying of the gas mixture resulting from stage i);
iii) optionally treatment of the gas mixture dried in stage ii) to remove the hydrogen and inert gases and to form a gas stream F11;
iv) distillation of the gas mixture dried in stage ii) or of said gas stream F11 resulting from stage iii) or of said stream F5.

The stream F5 resulting from stage B) or said stream resulting from stage A) employed in stage i) are preferably in gaseous form. The HCl and the HF are removed by passing either of said streams through water in a scrubbing column and then by scrubbing with a dilute base, such as NaOH or KOH. The remainder of the gas mixture, consisting of the reactants (H₂ and CTFE if present), dilution nitrogen (if present), trifluoroethylene and organic impurities, is directed to a dryer in order to remove the traces of scrubbing water.

Drying can be carried out using products such as calcium, sodium or magnesium sulfate, calcium chloride, potassium carbonate, silica gel or zeolites. In one embodiment, a molecular sieve (zeolite), such as siliporite, is used for the drying.

If the gas mixture, thus dried, comprises hydrogen or inert substances, stage iii) is preferably carried out. This can be carried out according to various techniques: absorption/desorption or membrane separation.

The gas mixture, thus dried, is optionally subjected to a stage of separation of the hydrogen and inert substances from the remainder of the other products present in the gas mixture by absorption/desorption in the presence of an alcohol comprising from 1 to 4 carbon atoms and preferably ethanol, at atmospheric pressure and at a temperature below ambient temperature, preferably of less than 10° C. and more preferably still at a temperature of −25° C., for the absorption. In one embodiment, the absorption of the organic substances is carried out in a countercurrent column with ethanol cooled to −25° C. The ethanol flow rate is adjusted according to the flow rate of organic substances to be absorbed. The hydrogen and inert gases, which are insoluble in ethanol at this temperature, are removed at the absorption column top. The organic substances are subsequently recovered in the form of said stream F11, by heating the ethanol to its boiling point (desorption).

Alternatively, the dried gas mixture obtained in stage ii) is subjected to stage (a) according to the first aspect of the present invention, if hydrogen and possibly nitrogen are present. This embodiment makes it possible to remove the hydrogen and possibly the nitrogen. Said dried gas mixture obtained in stage ii) is then brought into contact with said membrane M1 under the conditions described in this first aspect of the present invention. This embodiment is preferably obtained with said membrane M1 as described in the present patent application and which is made of a material selected from the group consisting of polyolefin, polyether, polyvinylidene fluoride, cellulose and polyimide, in particular made of a material selected from the group consisting of polypropylene, polymethylpentene, poly[oxy(2,6-dimethyl-1,4-phenylene)], poly(phenylene oxide), polyvinylidene fluoride, cellulose and polyimide, as explained above. The stream F11 obtained comprises trifluoroethylene and possibly chlorotrifluoroethylene. The stream F11 is then subjected to stage iv) or to stage B) to separate the trifluoroethylene and chlorotrifluoroethylene possibly present therein.

If the gas mixture dried in stage ii) does not contain hydrogen, inert compounds, stage iii) cannot be carried out, and the gas mixture dried in stage ii) is distilled in stage iv).

As mentioned above, said stream resulting from stage A) can be pretreated by stages i), ii) and optionally iii) which are described above before carrying out stage B) as described above. In this case, the stream F5 comprising trifluoroethylene is recovered and subjected to stage iv) to remove other organic compounds and to obtain high-purity trifluoroethylene. The stream F6 can be recovered and recycled in stage A).

According to stage iv), the gas mixture dried in stage ii) or said gas stream F11 obtained in stage iii), if it is carried out, or the stream F5 obtained in stage B), in particular when this is carried out after stages i), ii) and iii), is distilled to form and recover a stream F12 comprising trifluoroethylene. According to a preferred embodiment, the distillation stage iv) is carried out at a pressure of less than 3 bara, preferably at a pressure of between 0.5 and 3 bara, in particular at a pressure of between 0.9 and 2 bara. Carrying out a distillation at a pressure of less than 3 bara makes it possible to render the process more secure due to the explosive nature of trifluoroethylene above 3 bara. Said stream F12 is preferably recovered at the top of the distillation column. Before being recovered, the stream F12 can optionally be partially condensed at the top of the distillation column. When the partial condensation is carried out, the stream F12 is brought to a temperature of −50° C. to −70° C. The temperature is adjusted according to the pressure applied in stage iv). The partial condensation makes it possible to improve the efficiency of the distillation by limiting the content of additional compounds in the stream F12. The distillation of stage iv) also results in the formation of a stream F13 possibly comprising residual chlorotrifluoroethylene and organic impurities resulting from the hydrogenolysis reaction (stage A)). This stream F13 is generally recovered at the bottom of the distillation column. Said stream F13 can be recycled in stage A) after an optional purification treatment.

As explained above, in this fourth aspect, the present invention provides a process for separating the trifluoroethylene from the chlorotrifluoroethylene according to stage B) described above. Thus, the present invention provides a process for the separation of a mixture comprising trifluoroethylene and chlorotrifluoroethylene, said process comprising a stage of bringing said mixture into contact with said membrane M2 to form a stream F5 comprising the trifluoroethylene and a stream F6 comprising the chlorotrifluoroethylene.

Preferably, said membrane M2 is made of a material selected from the group consisting of polyolefin, polyether, polyimide, polymethyl methacrylate, cellulose and polyvinylidene fluoride. Preferably, said membrane M2 is made of a material selected from the group consisting of polypropylene, polymethylpentene, poly[oxy(2,6-dimethyl-1,4-phenylene)], poly(phenylene oxide), polyimide and cellulose acetate. This process is carried out according to the conditions described for stage B) above. This process is carried out with a membrane M2 as described in stage B).

Thus, in this fourth aspect, the present invention provides a process for the production of trifluoroethylene according to various embodiments. For example, the present invention provides a process for the production of trifluoroethylene in a reactor provided with a fixed catalytic bed comprising a catalyst, said process comprising:
A) a stage of reaction of chlorotrifluoroethylene with hydrogen in the presence of the catalyst and in the gas phase to produce a stream comprising trifluoroethylene and unreacted hydrogen and chlorotrifluoroethylene; and
B) a stage of bringing said stream resulting from stage A) into contact with a membrane M2 to form a stream F5 comprising trifluoroethylene and possibly hydrogen and a stream F6 comprising chlorotrifluoroethylene and possibly hydrogen;
i) the removal of HF and/or HCl from the stream F5 to form a gas mixture;
ii) the drying of the gas mixture resulting from stage i);
iii) optionally treatment of the gas mixture dried in stage ii) to remove the hydrogen and the inert gases and to form a gas stream F11;
iv) distillation of the gas mixture dried in stage ii) or of said gas stream F11 resulting from stage iii) to recover a stream F12 comprising trifluoroethylene.

The present invention also provides a process for the production of trifluoroethylene in a reactor provided with a fixed catalytic bed comprising a catalyst, said process comprising:
A) a stage of reaction of chlorotrifluoroethylene with hydrogen in the presence of the catalyst and in the gas phase to produce a stream comprising trifluoroethylene and unreacted hydrogen and chlorotrifluoroethylene;
i) the removal of HF and/or HCl from said stream resulting from stage A) to form a gas mixture;
ii) the drying of the gas mixture resulting from stage i);
iii) optionally treatment of the gas mixture dried in stage ii) to remove the hydrogen and the inert gases and to form a gas stream F11;
B) a stage of bringing said gas stream F11 or said gas mixture resulting from stage i) into contact with a membrane M2 to form a stream F5 comprising trifluoroethylene and possibly hydrogen and a stream F6 comprising chlorotrifluoroethylene and possibly hydrogen;
iv) distillation of said stream F5 to recover a stream F12 comprising trifluoroethylene.
   Separation of the Trifluoroethylene from a Hydrofluorocarbon

According to a fifth aspect, the present invention provides a process for the separation of a mixture comprising trifluoroethylene and a hydrofluorocarbon, said process comprising a stage of bringing said mixture into contact with a membrane M4 to form a stream F9 comprising trifluoroethylene and a stream F10 comprising said hydrofluorocarbon.

Preferably, said hydrofluorocarbon is a hydrofluoroalkane.

Preferably, said membrane M4 is made of a material selected from the group consisting of polyolefin, polyether, polyimide, polyaramid, polyamide, polysulfone, polyvinylidene fluoride, cellulose, polymethyl methacrylate, polytetrafluoroethylene, polyvinyl fluoride, polychlorotrifluoroethylene, polyethylene-tetrafluoroethylene and tetrafluoroethylene/perfluorovinyl ether copolymer optionally substituted by an SO₃H group. Preferably, said membrane M4 is made of a material selected from the group consisting of polyolefin, polyether, polyimide, polyvinylidene fluoride and cellulose.

According to a preferred embodiment, said membrane M4 is chosen from a film, a laminated structure, hollow fibers and coated fibers.

The term polyolefin refers in particular to polyethylene, polypropylene, polymethylpropene, polybutene, polypentene, polymethylpentene, polymethylbutene, polyhexene, polymethylpentene and polyethylbutene.

The term polyether refers in particular to a polyaryl ether comprising the monomeric unit —[—O—Ar—]— or —[—Ar¹—O—Ar²—]— in which Ar, Ar¹ and Ar² are, independently of one another, an aromatic ring comprising from 6 to 12 carbon atoms optionally substituted by one or more C₁-C₁₀ alkyl functional groups; preferably, Ar is a phenyl group optionally substituted by one, two, three or four C₁-C₃ alkyl functional groups. In particular, the polyether is poly[oxy(2,6-dimethyl-1,4-phenylene)] or poly(phenylene oxide).

Preferably, the cellulose is cellulose acetate.

Preferably, said membrane M4 is made of a material selected from the group consisting of polyolefin, polyether, polyimide and cellulose.

In particular, said membrane M4 is made of a material selected from the group consisting of polypropylene, polymethylpentene, cellulose acetate, polyimide, poly[oxy(2,6-dimethyl-1,4-phenylene)] and poly(phenylene oxide). In particular, said membrane M4 is made of polypropylene or of polymethylpentene.

According to a preferred embodiment, said hydrofluorocarbon is a hydrofluoroalkane selected from the group consisting of pentafluoroethane, 1,1,1,2-tetrafluoroethane, 1,1,2,2-tetrafluoroethane, 1,1-difluoroethane, 1,2-difluoroethane, 1,1,2-trifluoroethane, fluoromethane, difluoromethane, trifluoromethane, 1,1,1,2,2-pentafluoropropane, 1,1,1,2,3-pentafluoropropane, 1,1,1,3,3-pentafluoropropane and 1,1,1,2,3,3-hexafluoropropane.

Preferably, said hydrofluorocarbon is a hydrofluoroalkane selected from the group consisting of pentafluoroethane, 1,1,1,2-tetrafluoroethane, 1,1,2,2-tetrafluoroethane, 1,1-difluoroethane, 1,2-difluoroethane and 1,1,2-trifluoroethane.

Preferably, said membrane M4 has a selectivity of greater than 10, advantageously of greater than 15, preferably of greater than 20, more preferentially of greater than 25, in particular of greater than 30, said selectivity being calculated by the ratio of the permeability of the trifluoroethylene to the permeability of said hydrofluorocarbon through said membrane M4. In a particularly preferred embodiment, said hydrofluorocarbon is a hydrofluoroalkane selected from the group consisting of pentafluoroethane, 1,1,1,2-tetrafluoroethane and 1,1,2,2-tetrafluoroethane, and said membrane M4 is made of polypropylene, polymethylpentene, cellulose acetate, polyimide, poly[oxy(2,6-dimethyl-1,4-phenylene)] or poly(phenylene oxide). In a particularly preferred embodiment, said hydrofluorocarbon is a hydrofluoroalkane selected from the group consisting of pentafluoroethane, 1,1,1,2-tetrafluoroethane and 1,1,2,2-tetrafluoroethane and said membrane M4 is made of polypropylene or of polymethylpentene, preferably of polymethylpentene.

Thus, with respect to the starting mixture, the stream F9 is enriched in trifluoroethylene. For its part, the stream F10 is enriched in hydrofluorocarbon, with respect to the starting mixture. Preferably, the process is carried out at a pressure of 0.1 bara to 30 bara, advantageously of 0.2 bara to 25 bara, preferably of 0.3 bara to 20 bara, more preferentially of 0.4 bara to 15 bara, in particular of 0.5 bara to 10 bara, more particularly of 0.5 bara to 5 bara. During the implementation of the process, a pressure difference is observed between the inlet of the membrane and the outlet of the membrane. The differential pressure expressed here corresponds to the pressure difference existing between the inlet and the outlet of said membrane. Preferably, the differential pressure is from 1 to 3000 kPa, preferably from 50 to 2000 kPa, in particular from 100 to 1000 kPa, more particularly from 100 to 500 kPa. Preferably, the process is carried out at a temperature of 0° C. to 150° C., advantageously of 0° C. to 125° C., preferably of 5° C. to 100° C., more preferentially of 10° C. to 75° C., in particular of 10° C. to 50° C.

This process for the separation of trifluoroethylene from a hydrofluorocarbon can be integrated into an overall process for the production of trifluoroethylene. Thus, the present invention also provides a process for the production of trifluoroethylene comprising a stage A1) of dehydrofluorination of 1,1,1,2-tetrafluoroethane or of reaction between chlorodifluoromethane and chlorofluoromethane to form a stream comprising trifluoroethylene and 1,1,1,2-tetrafluoroethane and a stage B1) of separation of a stream comprising trifluoroethylene and a hydrofluorocarbon according to the fifth aspect of the present invention with a membrane M4 to form a stream F9′ comprising trifluoroethylene and a stream F10′ comprising said hydrofluorocarbon.

Preferably, said hydrofluorocarbon is a hydrofluoroalkane selected from the group consisting of pentafluoroethane, 1,1,1,2-tetrafluoroethane and 1,1,2,2-tetrafluoroethane, and said membrane M4 is made of a material selected from the group consisting of polypropylene, polymethylpentene, cellulose acetate, polyimide, poly[oxy(2,6-dimethyl-1,4-phenylene)] and poly(phenylene oxide).

In particular, said hydrofluorocarbon is 1,1,1,2-tetrafluoroethane and said membrane M4 is made of a material selected from the group consisting of polypropylene, polymethylpentene, cellulose acetate, polyimide, poly[oxy(2,6-dimethyl-1,4-phenylene)] and poly(phenylene oxide).

Stage A1) of production of trifluoroethylene can be a thermal dehydrofluorination of 1,1,1,2-tetrafluoroethane (HFC-134a) in the absence of catalyst or in the presence of a catalyst.

Pyrolysis of HFC-134a (Thermal Dehydrofluorination in the Absence of Catalyst) In the absence of catalyst, the dehydrofluorination of 1,1,1,2-tetrafluoroethane is carried out at a temperature of greater than 500° C., advantageously at a temperature of greater than 550° C., preferably at a temperature of greater than 600° C., more preferentially at a temperature of greater than 650° C., in particular of greater than 700° C., more particularly of greater than 800° C. The residence time is of between 0.1 and 100 seconds, advantageously between 0.1 and 75 seconds, preferably between 0.5 and 50 seconds, more preferentially between 0.5 and 10 seconds, in particular between 0.5 and 5 seconds. The pressure can be of between 1 bara and 50 bara, preferably between 1 bara and 25 bara, in particular between 1 bara and 10 bara. The reaction can be carried out in the presence of a diluent, such as nitrogen, helium or argon, preferably nitrogen.

In this embodiment, preferably, the output stream from stage A1) comprises, in addition to the trifluoroethylene, 1,1,1,2-tetrafluoroethane. The output stream from stage A1) can also comprise tetrafluoroethylene and 1,1,2,2-tetrafluoroethane. HF is also present in the output stream from stage A1). The HF can be removed before carrying out stage B1). The HF can be removed by ordinary techniques known to a person skilled in the art, such as sparging in water or an alkaline or caustic solution.

In this embodiment, stage B1) of the present process is carried out starting from a stream comprising trifluoroethylene, 1,1,1,2-tetrafluoroethane and possibly tetrafluoroethylene and 1,1,2,2-tetrafluoroethane. Stage B1) is carried out with a membrane M4 as defined above to form a stream F9′ comprising trifluoroethylene and a stream F10′ comprising 1,1,1,2-tetrafluoroethane. Preferably, said membrane M4 is made of a material selected from the group consisting of polypropylene, polymethylpentene, cellulose acetate, polyimide, poly[oxy(2,6-dimethyl-1,4-phenylene)] and poly(phenylene oxide). Stage B1) is carried out as indicated above in connection with the separation of the trifluoroethylene from a hydrofluorocarbon according to the fifth aspect of the present invention. Preferably, the 1,1,1,2-tetrafluoroethane is in anhydrous form. The term anhydrous is defined above in the present patent application.

### Catalytic Dehydrofluorination of HFC-134a

As indicated above, in another embodiment, stage A1) employs a dehydrofluorination of 1,1,1,2-tetrafluoroethane in the presence of a catalyst and in the gas phase. 1,1,1,2-Tetrafluoroethane is thus brought into contact in the gas phase with a catalyst based on a metal in the oxide, halide or oxyhalide form. The metal is selected from the group consisting of chromium, aluminum, cobalt, zinc, nickel, potassium, silver, cesium, sodium, calcium, titanium, vanadium, zirconium, molybdenum, tin, lead, magnesium and manganese. More particularly, the catalyst can be based on a metal in the oxide, fluoride or oxyfluoride form, said metal being selected from the group consisting of chromium, aluminum, cobalt, zinc, nickel, potassium, silver, cesium and sodium. Favorably, the catalyst is based on a metal in the oxide, fluoride or oxyfluoride form, said metal being selected from chromium and aluminum. The catalyst can be bulk (nonsupported) or supported on a support based on carbon (graphite, activated carbon) or on aluminum (alumina, fluorinated alumina, aluminum fluoride). The catalytic dehydrofluorination of HFC-134a is preferably carried out at a temperature of 50° C. to 500° C., advantageously of 100° C. to 450° C., preferably of 150° C. to 450° C., in particular of 200° C. to 450° C. The catalytic dehydrofluorination of HFC-134a is preferably carried out at a pressure of 1 bara to 20 bara, preferably of 1 bara to 15 bara, in particular of 3 bara to 10 bara. The catalytic dehydrofluorination of HFC-134a is preferably carried out at a contact time of 0.5 second to 60 seconds, preferably of 1 second to 45 seconds, in particular of 5 seconds to 30 seconds. In this embodiment of catalytic dehydrofluorination of HFC-134a, the output stream from stage A1) comprises, besides the trifluoroethylene, 1,1,1,2-tetrafluoroethane and HF. The output stream can also comprise one or more of the following compounds: 1,1,2,2-tetrafluoroethane (HFC-134), 2-chloro-1,1,1-trifluoroethane (HCFC-133a) or 2-chloro-1,1-difluoroethylene (HCFO-1122). The HF can be removed before carrying out stage B1). The HF can be removed by ordinary techniques known to a person skilled in the art, such as sparging in water or an alkaline or caustic solution.

In this embodiment, stage B1) of the present process is carried out starting from a stream comprising trifluoroethylene, 1,1,1,2-tetrafluoroethane and possibly 2-chloro-1,1,1-trifluoroethane, 2-chloro-1,1-difluoroethylene or 1,1,2,2-tetrafluoroethane. Stage B1) is carried out with a membrane M4 as defined above to form a stream F9′ comprising trifluoroethylene and a stream F10′ comprising 1,1,1,2-tetrafluoroethane. Preferably, said membrane M4 is made of a material selected from the group consisting of polypropylene, polymethylpentene, cellulose acetate, polyimide, poly[oxy(2,6-dimethyl-1,4-phenylene)] and poly(phenylene oxide). Stage B1) is carried out as indicated above in connection with the separation of the trifluoroethylene from a hydrofluorocarbon according to the fifth aspect of the present invention. Preferably, the 1,1,1,2-tetrafluoroethane is in anhydrous form. The term anhydrous is defined above in the present patent application.

### HCFC-22 and HCFC-31 Reaction

Alternatively, stage A1) is a stage of reaction between chlorodifluoromethane (HCFC-22) and chlorofluoromethane (HCFC-31), by thermal decomposition. The HCFC-22/HCFC-31 molar ratio is from 1:0.01 to 1:4.0, preferably from 0.1 to 1.5. Stage A1) is carried out at a temperature of 400° C. to 1200° C., preferably of 600° C. to 900° C., in particular of 710° C. to 900° C. Stage A1) is carried out at a pressure of 1 to 3.0 MPa, preferably of 1 to 1.5 MPa. The reactants, i.e. HCFC-22 and HCFC-31, can be heated and mixed prior to carrying out the reaction. The HCFC-22 can be heated to a temperature of 25° C. to 600° C., preferably of 100° C. to 500° C. The HCFC-31 can be heated to a temperature of 25° C. to 1200° C., preferably of 100° C. to 800° C. The contact time is from 0.01 to 10 seconds, preferably from 0.2 to 3.0 seconds. The output stream comprises, besides the trifluoroethylene, 1,1,1,2-tetrafluoroethane. The output stream can also comprise pentafluoroethane, 1,1,2,2-tetrafluoroethane or 2-chloro-1,1-difluoroethylene. The output stream can also comprise unreacted HCFC-22 and HCFC-31. HF can also be present in the output stream from stage A1). The HF can be removed before carrying out stage B1). The HF can be removed by ordinary techniques known to a person skilled in the art, such as sparging in water or an alkaline or caustic solution.

In this embodiment, stage B1) of the present process is carried out starting from a stream comprising trifluoroethylene, 1,1,1,2-tetrafluoroethane and possibly pentafluoroethane, 1,1,2,2-tetrafluoroethane, chlorofluoromethane, chlorodifluoromethane or 2-chloro-1,1-difluoroethylene. Stage B1) is carried out with a membrane M4 as defined above to form a stream F9′ comprising trifluoroethylene and a stream F10′ comprising 1,1,1,2-tetrafluoroethane. Preferably, said membrane M4 is made of a material selected from the group consisting of polypropylene, polymethylpentene, cellulose acetate, polyimide, poly[oxy(2,6-dimethyl-1,4-phenylene)] and poly(phenylene oxide). Stage B1) is carried out as indicated above in connection with the separation of the trifluoroethylene from a hydrofluorocarbon according to the fifth aspect of the present invention. Preferably, HCFC-22 and HCFC-31 are in anhydrous form. The term anhydrous is defined above in the present patent application.

Stage B1) is carried out whatever the reaction stage chosen (catalytic dehydrofluorination or dehydrofluorination in the absence of catalyst of HFC-134a or reaction of HCFC-22 with HCFC-31) with said membrane M4 as defined above according to the fifth aspect of the present invention.

In general, in the present invention, the processes for the production of trifluoroethylene are carried out in reactors which are resistant to corrosion (taking into account the presence of HF or HCl in the reaction streams). Stages A) or A1) can be carried out in reactors made of Monel or Inconel or Hastelloy or in reactors made of nickel.

### EXAMPLES

The permeability of a gaseous compound through a polymer is measured using an Evonik MET Crossflow Filtration Cell (with an internal diameter of 52 mm and an active surface area of 14 cm²) for the polymers in the form of a film or using a commercial module for the polymers in the form of fibers. The module made of PPO fiber is sold by Parker (reference module ST304—thickness of 50 μm for a surface area of 0.4 m²). The polyimide film is a Dupont Kapton HN film, the polymethylpentene film has the reference MX004, the silicone has the reference USP Class VI. The films are provided by Goodfellow.

In the examples below, with the exception of PPO, the membranes tested are in the form of a film with an active surface area of 14 cm² and the thickness of which is shown in table 1 below.

TABLE 1

| Material | Thickness |
|---|---|
| Polyimide | 25 μm |
| Cellulose acetate | 35 μm |
| PVDF | 25 μm |
| PMP | 50 μm |
| Polydimethylsiloxane | 250 μm |
| Polypropylene | 25 μm |

PMP = polymethylpentene;
PVDF = polyvinylidene fluoride

The permeability is calculated according to the following formula: P=Q×e×S⁻¹×ΔP⁻¹ with P: permeability in cm²·s⁻¹·Pa⁻¹
Q: permeate flow rate in cm³/s
e: thickness of the membrane in cm
S: surface area of the membrane in cm²
ΔP: pressure difference across the membrane in Pa (i.e. differential pressure mentioned in the present patent application)

The permeability is generally expressed in barrier (10⁻¹⁰·cm³(STP)·cm·cm²·s⁻¹·cm Hg⁻¹) according to the conversion:

$${P}_{barrer} = P \times {10}^{10} / ( 7.500615 \times {10}^{- 4} )$$

Thus, it is possible to calculate the permeability of a compound through a material from the data of the material (surface area, thickness), the pressure difference across the membrane and the measurement of the permeate flow rate through the membrane. The permeability is thus measured by keeping under pressure a compound upstream of the membrane in the absence of an outlet on the retentate side, and by measuring the flow rate of this same compound at atmospheric pressure on the permeate side. The tests are carried out at a temperature of 25° C., except for the silicone, the tests of which were carried out at 35° C. The tests are repeated several times, optionally at different pressures, to obtain a more accurate permeability value. Unless otherwise mentioned, the permeability remains constant whatever the ΔP (i.e. the pressure difference across the membrane).

### Example 1: Hydrogen/Fluorocarbon Separation

The experimental protocol detailed above was employed independently for each compound of the mixture under consideration: hydrogen and a fluorocarbon (trifluoroethylene (VF₃), 2,3,3,3-tetrafluoropropene (HFO-1234yf) or pentafluoroethane (HFC-125)). The membrane used is made of polypropylene, polymethylpentene, poly(phenylene oxide) (PPO), polyimide, PVDF or cellulose acetate. The results are shown in table 2 below. The permeability value is expressed in barrers. The selectivity mentioned in the table corresponds to the ratio of the permeabilities measured for the two entities under consideration.

TABLE 2

| | Permeability (barrer) | | | | Selectivity | | |
|---|---|---|---|---|---|---|---|
| | H₂ | VF₃ | HFO-1234yf | HFC-125 | H₂/VF₃ | H₂/1234yf | H₂/HFC-125 |
| PP | 6.2 | 0.5 | n.a. | n.a. | 12.4 | n.a. | n.a. |
| PMP | 169 | 14 | 2.6 | 0.2 | 12.1 | 65 | 847 |
| PPO | 105 × 10³ | 4.6 × 10³ | n.a. | n.a. | 23 | n.a. | n.a. |
| PVDF | 2.1 | 0.2 | n.a. | n.a. | 11 | n.a. | n.a. |
| PI | 3.5 | 0.2 | n.a. | n.a. | 17.5 | n.a. | n.a. |
| Cel. | 11 | 1 | n.a. | n.a. | 11 | n.a. | n.a. |

PP = polypropylene;
PMP = polymethylpentene;
PPO = poly(phenylene oxide);
n.a. = not applicable;
PI = polyimide;
PVDF = polyvinylidene fluoride;
Cel. = cellulose acetate

As is shown by the above data, the membranes made of polyolefins or polyether are more permeable to hydrogen than to fluorocarbons, both fluorocarbons of hydrofluoroolefins type and of hydrofluoroalkanes type. The membranes of polyolefin (polypropylene or polymethylpentene) or polyether (poly(phenylene oxide)) type thus make it possible to efficiently separate fluorocarbons, such as hydrofluoroolefins or hydrofluoroalkanes, from hydrogen.

### Example 2: Nitrogen/Fluorocarbon Separation

The experimental protocol detailed above in example 1 was employed independently for each compound of the mixture under consideration: nitrogen and a fluorocarbon (trifluoroethylene (VF₃), 2,3,3,3-tetrafluoropropene (HFO-1234yf) or pentafluoroethane (HFC-125)). The membrane used is made of silicone or polymethylpentene. The results are shown in table 3 below. The permeability value is expressed in barrers. The selectivity mentioned in the table corresponds to the ratio of the permeabilities measured for the two entities under consideration.

TABLE 3

| | Permeability (barrer) | | | | Selectivity | | |
|---|---|---|---|---|---|---|---|
| | N₂ | VF₃ | HFO-1234yf | HFC-125 | VF₃/N₂ | 1234yf/N₂ | N₂/HFC-125 |
| Silicone | 337 | 3045 | 3414 | n.a. | 9.1 | 10.1 | n.a. |
| PMP | 9.1 | n.a. | n.a. | 0.2 | n.a. | n.a. | 45.5 |

PMP = polymethylpentene;
n.a. = not applicable;
silicone = polydimethylsiloxane

As is shown by the above data, the N₂/HFC-125 selectivity, calculated by the ratio of the permeability of nitrogen through the PMP membrane to the permeability of HFC-125 through the PMP membrane (i.e. selectivity=9.1/0.2), is 45.5, which makes it possible to efficiently separate the two compounds. As is demonstrated by the present invention, membranes of polyolefin type (such as polymethylpentene) make it possible to efficiently separate hydrofluoroalkanes from nitrogen. In addition, membranes made of silicone (such as polydimethylsiloxane) are more permeable to hydrofluoroolefins than to nitrogen. A factor of 10 is observed between the permeability of nitrogen and that of hydrofluoroolefins, such as VF₃ or HFO-1234yf. The VF₃/N₂ and HFO-1234yf/N₂ selectivities obtained make it possible to confirm that membranes made of silicone efficiently separate hydrofluoroolefins (such as HFO-1234yf and VF₃) from nitrogen.

### Example 3: Process for the Production of Trifluoroethylene (VF₃)

Several compositions comprising from 29% to 44% of trifluoroethylene (VF₃), from 9% to 16% of chlorotrifluoroethylene (CTFE) and 37% hydrogen are brought into contact with a polyolefin membrane. The compositions are obtained following the implementation of a hydrogenolysis reaction between CTFE and hydrogen in the gas phase, in the presence of a palladium-on-alumina catalyst under the conditions described in the present patent application. The compositions also comprise organic impurities.

The permeability of the compositions is evaluated according to the protocol described above with a polymethylpentene membrane. The results are shown in table 4 below.

TABLE 4

| | Permeability (barrer) | | | Selectivity | | |
|---|---|---|---|---|---|---|
| | H₂ | VF₃ | CTFE | CTFE/VF₃ | CTFE/H₂ | H₂/VF₃ |
| PMP | 169 | 14 | 380 | 28 | 2 | 12.1 |

| | | | | VF₃/CTFE | H₂/CTFE | H₂/VF₃ |
|---|---|---|---|---|---|---|
| Cel. | 11 | 1 | <0.1 | >10 | >115 | 11 |
| PI | 3.5 | 0.2 | 0.01 | 15.1 | 264 | 17.5 |

PMP = polymethylpentene; PI = polyimide; Cel. = cellulose acetate; the permeability of the CTFE is a mean of two measurements: 387 barrer and 373 barrer, obtained respectively with a pressure difference between the inlet and the outlet of the membrane of 4.8 bar and 4.7 bar.

The above results show that polyolefin membranes are more permeable to hydrogen and to chlorotrifluoroethylene than to trifluoroethylene. Thus, a composition resulting from the hydrogenolysis reaction between CTFE and H₂ is easily separated by a membrane of polyolefin type, such as polymethylpentene. A large amount of CTFE and a large amount of hydrogen are withdrawn from the reaction stream and recycled. A stream enriched in trifluoroethylene is thus obtained. Alternatively, the above results show that membranes made of polyimide and of cellulose acetate are more permeable to hydrogen and to VF₃ than to chlorotrifluoroethylene. Thus, a composition resulting from the hydrogenolysis reaction between CTFE and H₂ is easily separated by a membrane of polyimide or cellulose type, such as cellulose acetate. A large amount of CTFE is removed from the reaction stream and recycled. A stream enriched in trifluoroethylene is also obtained, it being possible for the hydrogen to be easily separated from the trifluoroethylene by membrane separation as explained in the present patent application or by an absorption/desorption stage described above in the present patent application.

The high CTFE/VF₃ selectivity is particularly advantageous because the subsequent purification of the trifluoroethylene is more easily carried out in view of the low amount of CTFE in the trifluoroethylene stream resulting from the membrane separation stage.

### Example 4: VF₃/Hydrofluoroalkane Separation

The experimental protocol detailed above in example 1 was employed independently for each compound of the mixture under consideration: trifluoroethylene and a hydrofluorocarbon (pentafluoroethane (HFC-125) and 1,1,1,2-tetrafluoroethane (HFC-134a)). The membrane used is made of polymethylpentene. The results are shown in table 5 below. The permeability value is expressed in barrers. The selectivity mentioned in the table corresponds to the ratio of the permeabilities measured for the two entities under consideration.

TABLE 5

| | Permeability (barrer) | | | Selectivity | |
|---|---|---|---|---|---|
| | VF₃ | HFC-125 | HFC-134a | VF₃/HFC-125 | VF₃/HFC-134a |
| PMP | 14 | 0.2 | 0.3 | 72 | 47 |

PMP = polymethylpentene

The above results show that polyolefin membranes are more permeable to trifluoroethylene than to a hydrofluoroalkane, such as pentafluoroethane or 1,1,2,2-tetrafluoroethane. VF₃ can thus be easily separated from hydrofluoroalkanes, such as HFC-125 or HFC-134a. Thus, membranes of polyolefin type can be used in processes for the production of trifluoroethylene from 1,1,1,2-tetrafluoroethane or generating 1,1,1,2-tetrafluoroethane during the process. The desired product, i.e. VF₃, is efficiently separated from HFC-134a.

1-9. (canceled)

10. A process for the purification of a fluorocarbon from a mixture comprising said fluorocarbon and hydrogen, said process comprising a stage (a) of bringing said mixture into contact with a membrane M1 to form a stream F1 comprising said fluorocarbon and a stream F2 comprising the hydrogen.

11. The process as claimed in claim 10, characterized in that said membrane M1 is made of a material selected from the group consisting of polyolefin, polyether, polyimide, polyaramid, polyamide, polysulfone, polyvinylidene fluoride, cellulose, polymethyl methacrylate, polytetrafluoroethylene, polyvinyl fluoride, polychlorotrifluoroethylene, polyethylene-tetrafluoroethylene and tetrafluoroethylene/perfluorovinyl ether copolymer optionally substituted by an SO₃H group.

12. The process as claimed in claim 10, characterized in that the fluorocarbon is selected from the group consisting of fluoromethane, difluoromethane, trifluoromethane, trifluoroethylene, 1,1-difluoroethylene, 1,2-difluoroethylene, tetrafluoroethylene, chlorotrifluoroethylene, fluoroethane, pentafluoroethane, 1,1,1,2-tetrafluoroethane, 1,1,2,2-tetrafluoroethane, 1,1-difluoroethane, 1,2-difluoroethane, 1,1,2-trifluoroethane, 1,1,1-trifluoroethane, 2-chloro-1,1,2-trifluoroethane, 1-chloro-1,1,2-trifluoroethane, 2-chloro-1,1,1-trifluoroethane, 3,3,3-trifluoropropene, hexafluoropropene, 1,1,1,3,3,3-hexafluoropropane, 1,1,2,2,3,3-hexafluoropropane, 1,1,1,2,2,3-hexafluoropropane, 1,1,1,2,3,3-hexafluoropropane, 1,1,2,2,3-pentafluoropropane, 1,1,1,2,2-pentafluoropropane, 1,1,2,3,3-pentafluoropropane, 1,1,1,2,3-pentafluoropropane, 1,1,1,3,3-pentafluoropropane, 2-chloro-1,1,1,2-tetrafluoropropane, 3-chloro-1,1,1,3-tetrafluoropropane, 2,3-dichloro-1,1,1-trifluoropropane, 1,1,3,3,3-pentafluoropropene, 1,1,2,3,3-pentafluoropropene, 1,2,3,3,3-pentafluoropropene, 2-chloro-3,3,3-trifluoropropene, 1-chloro-3,3,3-trifluoropropene, 2,3,3,3-tetrafluoropropene, 1,3,3,3-tetrafluoropropene, 1,1,2,3-tetrafluoropropene, 1,1,3,3-tetrafluoropropene, 1,2,3,3-tetrafluoropropene, 1,1,3-trifluoropropene, 1,1,2-trifluoropropene, 3,3,3-trifluoropropene, 1,2,3-trifluoropropene, 2,3,3-trifluoropropene, 1,3,3-trifluoropropene, 1,1-difluoropropene, 1,2-difluoropropene, 2,3-difluoropropene and 3,3-difluoropropene.

13. The process as claimed in claim 10, characterized in that the fluorocarbon is selected from the group consisting of trifluoroethylene, 1,1-difluoroethylene, 1,2-difluoroethylene, tetrafluoroethylene, chlorotrifluoroethylene, pentafluoroethane, 1,1,1,2-tetrafluoroethane, 1,1,2,2-tetrafluoroethane, 1,1-difluoroethane, 1,2-difluoroethane, 1,1,2-trifluoroethane, 2-chloro-1,1,2-trifluoroethane, 1-chloro-1,1,2-trifluoroethane, 2-chloro-1,1,1-trifluoroethane, difluoromethane, trifluoromethane, 2,3,3,3-tetrafluoropropene, 1,3,3,3-tetrafluoropropene, 1-chloro-3,3,3-trifluoropropene, 2-chloro-3,3,3-trifluoropropene, 1,1,1,2,2-pentafluoropropane, 1,1,1,2,3-pentafluoropropane, 1,1,1,3,3-pentafluoropropane, 3-chloro-1,1,1,3-tetrafluoropropane, 2-chloro-1,1,1,2-tetrafluoropropane, 2,3-dichloro-1,1,1-trifluoropropane, hexafluoropropene, 1,2,3,3,3-pentafluoropropene, 3,3,3-trifluoropropene and 1,1,1,2,3,3-hexafluoropropane.

14. The process as claimed in claim 10, characterized in that the fluorocarbon is selected from the group consisting of trifluoroethylene, 1,1-difluoroethylene, 1,2-difluoroethylene, tetrafluoroethylene, pentafluoroethane, 1,1,1,2-tetrafluoroethane, 1,1,2,2-tetrafluoroethane, 1,1-difluoroethane, 1,2-difluoroethane, 1,1,2-trifluoroethane, difluoromethane, trifluoromethane, 2,3,3,3-tetrafluoropropene, 1,3,3,3-tetrafluoropropene, 1,1,1,2,2-pentafluoropropane, 1,1,1,2,3-pentafluoropropane, 1,1,1,3,3-pentafluoropropane, hexafluoropropene, 1,2,3,3,3-pentafluoropropene, 3,3,3-trifluoropropene and 1,1,1,2,3,3-hexafluoropropane.

15. The process as claimed in claim 10, characterized in that said membrane M1 has a selectivity of greater than 5, said selectivity being calculated by the ratio of the permeability of the hydrogen to the permeability of said fluorocarbon through said membrane M1.

16. The process as claimed in claim 10, characterized in that said membrane M1 is made of a material comprising polypropylene or polymethylpentene or poly[oxy(2,6-dimethyl-1,4-phenylene)] or poly(phenylene oxide) or cellulose or polyimide or polyvinylidene fluoride (PVDF).

17. The process as claimed in claim 10, characterized in that said mixture and said stream F1 also comprise nitrogen, said process comprising a stage (b) of bringing said stream F1 into contact with a membrane M1′ to form a stream F3 comprising said fluorocarbon and a stream F4 comprising nitrogen.

18. The process as claimed in claim 17, characterized in that said membrane M1′ is made of a material comprising polypropylene, polymethylpentene or polyalkylsiloxane.
